(19) United States
(12) Patent Application Publication
(10) Pub. No.: US 2026/0242814 A1
(43) Pub. Date: Aug. 20, 2026

(54) WHEAT TRANSGENIC EVENT IND-ØØ412-7

(71) Applicant: Bioceres LLC, Wilmington, DE (US)

(72) Inventors: Patricia MIRANDA, Rosario (AR); Martin VAZQUEZ, Rosario (AR); Carlos DEZAR, Rosario (AR); Francisco AYALA, Rosario (AR); Geronimo WATSON, Rosario (AR)

(73) Assignee: Bioceres LLC, Wilmington, DE (US)

(21) Appl. No.: 19/181,953

(22) Filed: Apr. 17, 2025

Related U.S. Application Data
(62) Division of application No. 17/795,343, filed on Feb. 5, 2020, now Pat. No. 12,305,180.

(51) Int. Cl. C12N 15/82; C07K 14/415

(52) U.S. Cl. CPC C12N 15/8271; C07K 14/415

(57) ABSTRACT

The present invention refers to a wheat plant, seed or part of it comprising event IND-ØØ412-7, a commodity product resulting from the seed, a recombinant DNA molecule comprised in event IND-ØØ412-7, DNA primers and probes useful for detecting event IND-ØØ412-7, DNA detection kit, method for producing a wheat plant tolerant to abiotic stresses and method for detecting the presence of DNA belonging to wheat event IND-ØØ412-7 in a sample.

### RELATED APPLICATIONS

The present patent document is a divisional application of U.S. patent application Ser. No. 17/795,343, filed Jul. 26, 2022, which is 371 application of PCT Application Serial No. PCT/US2020/016832, filed Feb. 5, 2020, which are hereby incorporated by reference.

### REFERENCE TO APPENDIX [CD ROM/SEQUENCE LISTING]

This application contains a Sequence Listing XML which has been electronically submitted in .xml format. The .xml file is named “238419_Sequence_listing.xml,” was created on Apr. 14, 2025 and is 160,351 bytes in size. The sequence listing contained in this .xml file is part of the specification and hereby incorporated by reference in its entirety.

### FIELD OF THE INVENTION

The invention relates to the fields of plant production, plant breeding and agriculture. More specifically, it relates to wheat transgenic event IND-ØØ412-7, nucleotide sequences, plants, parts of plants, seeds, cells, agricultural products, and detection and production methods related to wheat transgenic event IND-ØØ412-7.

### BACKGROUND OF THE INVENTION

Improving crop yields and their characteristics has become essential to satisfy food demand. Due to biotechnological developments combined with agriculture, new crops have been developed with the capacity to adapt to diverse environmental and/or ecological conditions.

While grown, plants are exposed to a variety of abiotic stresses: drought, salinity, low and high temperatures, excessive radiation, low nutrient availability, soil compaction that prevents root development, etc. (Duque et al., 2013; Sayed, 2003). All of them can affect at some point plant growth and development, as well as yield. It is reasonable to suppose that any of these environmental factors may occur during crop life cycles in the field. When this is the case, complex response mechanisms are triggered, which will be later on reflected in the measurements performed, as they result from the integration of such stress effects.

One of the most commonly used techniques to mitigate environmental negative effects on crops is based on transgenic events, i.e., the insertion of genes of interest in the genome of a target crop. However, production and selection of a commercially suitable transgenic event requires extensive investigation, analysis and characterization of a large number of individual transformation events. This makes it possible to select an event containing the desired trait, able to develop the phenotypic and agricultural characteristics required for such event to be suitable for commercial purposes, with no negative effect on other crop features.

This process requires the generation of transgenic events that will be molecularly and phenotypically characterized in order to identify and select the event expressing the heterologous gene of interest considering the desired phenotype to be obtained.

Event selection implies stages of laboratory development as well as tests carried out under controlled conditions either in the field and/or in greenhouses. It is necessary to analyze the response to events along the years, in multiple locations and under a variety of environmental conditions in order to select the event that meets the required phenotypic and genetic characteristics as well as the commercial features required.

The present invention features this type of commercially suitable event, which enables new advantageous traits in wheat.

There is a wide variety of genes that may be used to generate commercially suitable events. A gene whose expression is of special interest is the one that encodes transcription factor HAHB4.

HAHB4 (which stands for Helianthus Annuus Homeobox-4) is a sunflower transcription factor that belongs to the HD-Zip family. The HaHB4 gene expression is regulated at transcriptional level by external environmental factors, such as water availability and soil salinity, as well as the phytohormones related to these factors, abscisic acid and ethylene.

Patent AR81216B2 discloses gene HaHB4, inducible by water deficit and abscisic acid and encoding a sunflower transcription factor type HD-Zip. This patent also discloses gene isolation and characterization, and its introduction in the model plant Arabidopsis thaliana. However, it reports neither transgenic plants of commercial interest carrying the event of the present invention nor their advantageous properties regarding concomitant abiotic stresses that occur in agriculture conditions.

In patent AR81216B2 no mention is made either concerning the particular selection of an event expressing gene HaHB4 in order to preserve the main trait of interest, i.e., drought tolerance, with no impact on other agricultural features.

Furthermore, application AR090110A1 discloses modified gene HaHB4, particularly HaHB4.2, inducible by water deficit and abscisic acid and encoding a modified sunflower transcription factor type HD-Zip, particularly mod1HaHB4. This publication discloses generation and characterization of modified HaHB4.2 expression constructs and their introduction in model plant Arabidopsis thaliana. In broad terms, this publication also discloses generation and selection of soybean, wheat and corn transgenic events containing gene HaHB4.2.

### DETAILED DESCRIPTION OF THE INVENTION

The present invention provides wheat plants resistant to abiotic stress containing event IND-ØØ412-7. These plants present advantages for their growth in unfavorable environmental conditions, thus allowing a higher yield.

More specifically, the present invention refers to the wheat event named IND-ØØ412-7, which has a representative seed registered in the American Type Culture Collection (ATCC) under access number PTA-126141 together with its progeny.

The present invention also includes wheat plants comprising event IND-ØØ412-7 represented by sequences SEQ ID No.: 1 and SEQ ID No.: 2.

The transgenic insert present in the event of the invention and in the seed registered comprises the selectable bar marker-gene and the gene HaHB4, which provides tolerance to abiotic stress. The bar gene derived from Streptomyces hygroscopicus, encodes PAT (Phosphinothricin Acetyl Transferase) protein. Gene HaHB4 derives from the sunflower plant, sp. Helianthus annuus, and encodes for a protein type HD-Zip I, which has a protein domain of homeodomain-type associated to a leucine zipper that provides abiotic stress-tolerance, mainly to drought. Regulation of genes of interest may be directed by different promoter sequences with different expression levels, sensitivity and tissue specificity. Experts in this field know that any promoter or nucleic acid terminator that directs or regulates the expression of a gene of interest may be used with no impact on the essence of the invention. Particularly, the event developed in the present invention contains the corn ubiquitin 1 gene promoter (prUBI-1) and the Tnos terminator for the bar gene that gives resistance to glufosinate ammonium herbicide. Furthermore, the event comprises once again the corn ubiquitin 1 gene promoter (prUBI-1) and the Tnos terminator to regulate the expression of the HaHB4 encoding region (FIG. 1 and FIG. 2).

Other aspects of the invention comprise the progeny of wheat plants, seeds and/or renewable parts of plants, seeds and progeny comprising wheat event IND-ØØ412-7, as well as food products for human or animal consumption derived from them. The invention also includes parts of plants comprising event IND-ØØ412-7, including, without limitation, pollen, ovules, flowers, shoots, roots, leaves, cell vegetative nuclei and other plant cells comprising event IND-ØØ412-7. The invention also refers to wheat plants comprising wheat event IND-ØØ412-7 featuring tolerance to multiple abiotic stresses, such as drought, salinity, low and high temperatures, radiation excess, low nutrient availability, soil compaction, etc., and different combinations of these factors.

The present invention partially refers to the cultivation of plants tolerant to abiotic stresses. In addition, it includes a novel event of wheat plant transformation comprising polynucleotides, as described herein, which are inserted in specific sites within the wheat genome, providing specific genetic and phenotypic features.

In some embodiments, this event/polynucleotide may be “stacked” with other traits including, for example, agronomic traits, and herbicide and/or insect tolerance. However, the present invention includes plants containing the individual event, as described herein.

Additional traits may be stacked within the plant genome or in the same locus as event IND-ØØ412-7, for example, by means of plant crossing, retransformation of the transgenic plant containing event IND-ØØ412-7 or addition of new traits through integration directed by homologous recombination.

In an embodiment, the present invention includes two wheat chromosomal sites located in chromosome 2. In some embodiments, the directed sites comprise heterologous nucleic acids. Wheat chromosomal sites are located between flanking sequences defined in FIG. 3 (SEQ ID Nos: 7, 8, 9 and 10).

In an embodiment, the present invention includes a method for producing wheat transgenic plants, comprising the insertion of a heterologous nucleic acid in one or several specific positions within chromosome 2.

In particular, the method comprises transforming a cell or cell culture with DNA sequences described in SEQ ID No.: 1 and SEQ ID No.: 2 and regenerate the cell in a stable way to originate a new whole plant.

Transformation of this plant cell may be performed through diverse techniques, whether physical, viral or chemical, including, among others, bio-ballistics, electroporation, bacterial transformation, or a combination of these techniques. All of them are well known by the expert in the field.

In particular, this invention uses a gene gun to boost DNA plasmid molecules with the sequences of interest (FIG. 2), more precisely, bombarded with plasmids pIND4-HB4 and pIND4-BAR (FIG. 1 and FIG. 2; SEQ ID No: 3 and SEQ ID No: 4).

Furthermore, the present invention provides tests for detecting the presence of this event in a wheat sample. The tests can be based on the DNA sequence of the recombinant constructs inserted within the wheat genome and on the genomic sequences flanking the insertion site. Kits and conditions useful to carry out the tests are also provided.

Therefore, the present invention partially refers to the cloning and analysis of DNA sequences belonging to all the inserts or to part of them and to the flanking regions (in transgenic wheat lines). These sequences are unique. Event-specific primers may be generated based on these inserts and on the flanking (and union) sequences. The PCR technique proved that these events can be identified through the analysis of amplicons generated by these sets of event-specific primers. Therefore, these and other related procedures may be used to identify wheat lines comprising the present invention event in an unequivocal manner.

The present invention also refers to PCR assay tests. These tests include, among others, quantitative real time (qPCR) or end-point PCR, to detect IND-ØØ412-7 event, amplicons and fragments thereof.

The invention also presents DNA molecules comprising a sufficient portion of the contiguous nucleotide sequence of SEQ ID No.: 5 and SEQ ID No.: 6 for it to operate as a DNA probe hybridizing under stringent conditions to DNA molecules comprising a nucleotide sequence selected from the group consisting of SEQ ID No.: 5 and SEQ ID No.: 6, and not hybridizing under stringent conditions to a DNA molecule not comprising a nucleotide sequence selected from the group consisting of SEQ ID No.: 5 and SEQ ID No.: 6.

In some instances, the probes used may be labeled with molecules that send out a detectable signal. An example of such molecules are fluorochromes. That is, oligonucleotides presenting fluorochromes at both ends and having a sequence complementary to part of the DNA fragment to be amplified. Among others, FAM, TET, HEX, JOE, CAL Fluor® Quasar®, and Pulsar® dyes.

The invention also discloses a pair of DNA molecules consisting of a first DNA molecule and a second DNA molecule different from the first one, in which each of the first and second DNA molecules comprise a sufficient portion of contiguous nucleotides of SEQ ID No.: 5 and SEQ ID No.: 6 to operate as DNA probes if used together in an amplification reaction with DNA derived from event IND-ØØ412-7 to produce a diagnostic DNA amplicon for wheat transgenic event IND-ØØ412-7 in a sample.

The invention also describes a method for detecting the presence of DNA obtained from event IND-ØØ412-7 in a sample. The method comprises the comparison of the sample with the DNA molecules used as probe and primers, subjecting them to the same stringent hybridization conditions, and then detecting hybridization of the DNA probe to DNA in the amplified sample with the use of specific primers. This hybridization indicates the presence of DNA derived from the wheat transgenic event IND-ØØ412-7 in this sample.

The invention further presents a method for detecting the presence of a DNA molecule obtained from wheat transgenic event IND-ØØ412-7 in a sample, comparing the DNA preparation derived from this sample with a pair of oligonucleotides used as primers to produce an amplification reaction sufficient to produce a DNA amplicon comprising a sequence selected from the group that consists of SEQ ID No.: 5 and SEQ ID No.: 6, in order to detect the presence of the DNA amplicon in the reaction, where the presence of the DNA amplicon indicates the presence of a DNA molecule derived from IND-ØØ412-7 in the sample.

In addition, the invention presents a DNA detection kit comprising at least one DNA molecule with sufficient number of contiguous nucleotides of SEQ ID No.: 5 and SEQ ID No.: 6 for it to operate as a primer or specific DNA probe to detect the presence of DNA derived from wheat transgenic event IND-ØØ412-7, where DNA detection proves the presence of this event in a sample.

The invention also features a wheat plant, seed, cell, or a part of this plant comprising a nucleic acid molecule selected from the group consisting of SEQ ID No.: 5 and SEQ ID No.: 6. The invention further presents a wheat plant, seed, cell, or part of a plant tolerant to abiotic stresses. In addition, the invention features a wheat plant, seed, cell or part of a plant, whose genome produces an amplicon comprising a DNA molecule selected from the group consisting of SEQ ID No.: 5 and SEQ ID No.: 6 when tested with a DNA amplification method.

The invention further presents a wheat plant or seed, where the wheat plant or seed is generated from wheat transgenic event IND-ØØ412-7, or is a hybrid or heterozygous having at least one parent derived from wheat transgenic event IND-ØØ412-7.

The invention further presents a non-living plant material comprising a recombinant DNA molecule selected from the group consisting of SEQ ID No.: 5 and SEQ ID No.: 6.

In addition, the invention presents a commodity product resulting from wheat transgenic event IND-ØØ412-7 that comprises a nucleic acid molecule selected from the group consisting of SEQ ID No.: 5 and SEQ ID No.: 6, where detection of a nucleotide sequence in a sample derived from the commodity product defines that the commodity product derives from wheat transgenic event IND-ØØ412.7. The invention further presents a commodity product selected from the group consisting of whole or processed seeds, meal, flour, flakes, bioethanol, biogas, or other biomaterials, among others. The invention also features a method for producing a commodity product by obtaining a wheat plant or a part of it comprising wheat transgenic event IND-ØØ412-7 and for producing a wheat commodity product from the wheat plant or a part of it.

The invention presents a method for producing a wheat plant tolerant to abiotic stresses by crossing a plant with wheat transgenic event IND-ØØ412-7 comprising a nucleic acid molecule selected from the group consisting of SEQ ID No.: 5 and SEQ ID No.: 6 with a second wheat plant, in order to produce seeds, collect the seeds resulting from the cross, grow the seeds to produce multiple progeny plants, and select a progeny plant tolerant to abiotic stresses.

### BRIEF DESCRIPTION OF THE DRAWINGS

FIG. 1: Schemes of plasmids pIND4-HB4 and pIND-Bar used in IND-ØØ412-7 obtaining. In the schemes are shown the CDSs of HaHB4 (left) and bar (right) with their regulatory elements and plasmid backbones. prUbi-1: maize Ubi-1 gene promoter region. prUbi-1 Exon: maize Ubi-1 gene first exon. prUbi-1 Intron: maize Ubi-1 gene first intron. HaHB4: CDS of HaHB4. Bar: CDS of bar. Tnos: terminator sequence of nos gene. pBR322 origin: ColE1 replication origin. bla: bla gene.

FIG. 2: Plasmid Sequences and Sequences of Interest.

FIG. 3: Resulting junction point sequences (JPs) supported by illumina and PacBio reads, and by PCR amplification products sequenced by Sanger. From the JPs, full obtained Wheat sequences are shown while only first 100 bp for the plasmid. Numbers represent absolute position on the Insert sequences.

FIG. 4: Yield of transgenic lines (events) and the parental control (wild type) at six field trials conducted through 2009 to 2011. Thin bars represent the standard error of the mean (SEM).

FIG. 5: Southern blots of IND-ØØ412-7 plant DNA digested with HindIII, BamHI and AseI. Blots were hybridized with DIG-labeled probes for HaHB4 detection. DNA bands in IND-ØØ412-7 digests hybridizing to probes are pointed with white arrows. HB4 Soybean transgenic plant (IND-ØØ41Ø-5) was used as positive and hybridizing band is pointed with red arrow. DIG-labeled Marker VII and III ladder band sizes are indicated on the left and right, respectively, of the blots in kb.

FIG. 6: Southern blots of IND-ØØ412-7 plant DNA digested with HindIII, BamHI and AseI. Blots were hybridized with DIG-labeled probes for bar detection. DNA bands in IND-ØØ412-7 digests hybridizing to probes are pointed with white arrows. HB4 Soybean transgenic plant (IND-ØØ41Ø-5) was used as positive and hybridizing band is pointed with red arrow. DIG-labeled Marker VII and III ladder band sizes are indicated on the left and right, respectively, of the blots in kb.

FIG. 7: Evaluation of integrity and size distribution of the DNA sample produced by chromosome 2D sorting. Average fragment size 5951 bp.

FIG. 8: PacBio read length distribution. Histogram representing absolute frequency (number of reads) in intervals of 1000 bp read length.

FIG. 9: Quality scores of PacBio reads. The upper graph represents the quality scores of base calls as a function of the read position in percentage and the lower graph as a function of the read position in bp.

FIG. 10: DNA sequence schemes of IND-ØØ412-7 long insert containing HaHB4 (red) and bar (light blue) complete (c) and incomplete (i) coding regions. This insert is subdivided in four fragments for their better understanding. a) HaHB4 incomplete (i) coding region is located between two incomplete fragments of Ubi-1 intron. Besides there are two bar coding regions, complete (c) and incomplete (i). The bar (i) is between two Tnos region. Bar (c) is located between its regulatory elements in the correct positions. b) HaHB4 (c) coding region is located downstream of the proximal region of ubi-1 intron and the 5′ promoter region and upstream its Tnos. Bar (i) is located between two intron ubi-1 regions. c) Bar (c) is located between an invert Ubi-1 promoter region and a Tnos region. Bar (i) is located between ubi-1 parcial promoter region and bla gene. d) Two bar (c) coding region are located between their complete regulatory elements.

FIG. 11: DNA sequence schemes of IND-ØØ412-7 short insert containing HaHB4 and bar complete (c) coding region. This insert is subdivided in three fragments for their better understanding. b) Bar (c) is located between an invert and short sequence prGBL-1 region and a Tnos region. c) HaHB4 complete (c) coding region is located between incomplete fragment ofUbi-1 intron and Tnos.

FIG. 12: Coverage amongst JPs blasted against T. aestivum (at http://plants.ensembl.org/).

### BRIEF DESCRIPTION OF THE SEQUENCES

SEQ ID No. 1: Long insert without flanking regions
SEQ ID No. 2: Short insert without flanking regions
SEQ ID No. 3: pIND4+HB4
SEQ ID No. 4: pIND4+BAR
SEQ ID No. 5: Long insert with flanking regions
SEQ ID No. 6: Short insert with flanking regions
SEQ ID No. 7: 5′ flanking region (long insert)
SEQ ID No. 8: 3′ flanking region (long insert)
SEQ ID No. 9: 5′ flanking region (short insert)
SEQ ID No. 10: 3′ flanking region (short insert)
SEQ ID No. 11: HB4 forward primer
SEQ ID No. 12: HB4 reverse primer
SEQ ID No. 13: Bar forward primer
SEQ ID No. 14: Bar reverse primer

### DETAILED DESCRIPTION

The following definitions and methods are presented to better define the invention and to guide those trained in this technique to put it in practice. Unless otherwise stated, the terms used here are to be construed with the same meaning conventionally given to them by those trained in the relevant technique.

### EXAMPLES

### Example 1: Construction of Plasmids pIND4-HB4 and pIND4-Bar

The vectors used to obtain wheat IND-ØØ412-7 are based on a series of plasmids that use the corn ubiquitin promoter to direct the expression of genes in plants (Christensen and Quail, 1996). This promoter has proved to be very efficient in monocotyledons (Christensen et al., 1992). These plasmids use vector base pUC8 to perform the cloning and have been generated as a result of cloning the Pst1 fragment of the Ubi-1 corn ubiquitin gene of 1992 pb. This fragment is the functional promoter and consists of: 899 pb of the sequence of gene Ubi-1, a sequence of 83 pb of the 5′-untranslated region from the first exon and the sequence of 1010 pb of the first intron, ending in the reconstruction of the Pst1 site. The original intron, present in the 5′-untranslated region of gene Ubi-1 was retained in all these vectors since previous studies revealed that introns stimulate the expression of transgenes in cereal plants (Vasil et al., 1993).

Two plasmids based on this series, pIND4-HB4 y e1 pIND4-Bar, have been used in the genetic modification present in IND-ØØ412-7.

pIND4-HB4 (SEQ ID NO:3)

This plasmid has a polycloning site from which site BamHI was used to clone the encoding region of gene HaHB4. This restriction site is located after the sequences of the corn Ubi-1 gene promoter, the first exon and the first intron of the gene. This construct prUbi-1/Ubi1-Exon/Ubi-1Intron/HaHB4 was designed to obtain the constitutive expression of HAHB4. After the polycloning site and after the CDS of HaHB4, this vector has a sequence of the nopaline-synthase gene terminator of A. tumefaciens which includes the polyadenylation signal.

pIND4-Bar (SEQ ID NO: 4)

This plasmid has also been constructed by binding Ubi-1 regulatory regions to the encoding region of the bar gene of S. hygroscopicus in the BamHI site. This construct, prUbi-1/Ubi1-Exon/Ubi-1 Intron/bar, allows selection of the transgenic plants expressing PAT and tolerate glufosinate ammonium herbicides, which become inactive due to this enzyme (Thompson et al., 1987). After the bar encoding region, this vector also contains the nopaline-synthase gene terminator of A. tumefaciens, which includes the polyadenylation signal.

### Example 2: Transformation of Wheat Plants and Selection of Event IND-ØØ412-7

The co-transformation method used to obtain event IND-ØØ412-7 was based on the methods developed by Barcelo and Lazzeri (1995), Pastori et al. (2001), and Rasco-Gaunt et al. (2001). These methods are also used in several research centers.

Firstly, the wheat kernels were harvested and sterilized. The kernels were collected from the spikelets and were sterilized. Then, the embryos were isolated and their axes were removed to avoid early germination. The scutella were placed in the appropriate medium, in the middle of a Petri plate, with the undamaged region of the explant facing upwards for it to experience the impact of the microparticles.

For bombardment, the gold particles covered with DNA from both plasmids of interest, pIND4-HB4 y pIND4-Bar, were boosted inside the cells of the explants using gene gun PDS-1000/He (Biolistic® PDS-1000/He Particle Delivery System, BIO-RAD).

After bombardment, the explants treated were placed in the fresh culture medium under the hormonal conditions necessary for the generation of the embryogenic calli. The selection process started three weeks later, once the embryos were already developed. The selection agent used in this case was glufosinate ammonium.

The seedlings that survived selection rounds due to the PAT expression and developed a good root system were transferred and placed in a growth chamber. At this point, samples were taken to prove the presence of bar and HaHb4 using PCR. Once the plants were developed, they were transferred to bigger pots where they were grown until harvest.

### Event Selection

Transgenic plants were generated following a biolistic protocol using the British variety Cadenza. The first multiplication (T1 seed) was carried in a growth chamber between August and December of 2007. Approximately 20 individuals derived from each transgenic event were sampled for a segregation test by PCR determination (Table 1).

TABLE 1
Segregation test. Lines with p-values > 0.15 were
carried to next generation.

| | PCR | | | | |
|---|---|---|---|---|---|
| Line | Positive | Negative | n | X² (3:1) | p-value |
| Ta.IV.ii.a.1 | 2 | 14 | 16 | 33.33 | 0.00 |
| Ta.IV.ii.a.2 | 9 | 8 | 17 | 4.41 | 0.04 |
| Ta.IV.ii.a.3 | 14 | 6 | 20 | 0.27 | 0.61 |
| Ta.IV.ii.a.4 | 12 | 7 | 19 | 1.42 | 0.23 |
| Ta.IV.ii.a.5 | 8 | 10 | 18 | 8.96 | 0.00 |
| Ta.IV.ii.a.6 | 17 | 2 | 19 | 2.12 | 0.15 |
| Ta.IV.ii.a.7 | 3 | 15 | 18 | 32.67 | 0.00 |
| Ta.IV.ii.a.8 | 7 | 11 | 18 | 12.52 | 0.00 |
| Ta.IV.ii.a.9 | 5 | 15 | 20 | 26.67 | 0.00 |
| Ta.IV.ii.a.10 | 5 | 15 | 20 | 28.12 | 0.00 |
| Ta.IV.ii.a.11 | 15 | 5 | 20 | 0.00 | 1.00 |
| Ta.IV.ii.a.12 | 14 | 4 | 18 | 0.07 | 0.79 |

Lines derived from selfings of selected events (3:1 segregation in T1) were sowed in August of 2008 under a hail shelter. Growth and development of these lines were characterized throughout the growing season (Table 2).

TABLE 2
Phenology.

| | Days after sowing | | | | | | | | | | | | | | | | | |
|---|---|---|---|---|---|---|---|---|---|---|---|---|---|---|---|---|---|---|
| Line | 12 | 20 | 27 | 34 | 42 | 49 | 57 | 63 | 70 | 77 | 84 | 91 | 98 | 105 | 111 | 119 | 126 | 131 |
| | --------------------------------- Zadocks scale value --------------------------------- | | | | | | | | | | | | | | | | | |
| Wild Type | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Wild Type | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.1.2 | 1.1 | 1.3 | 1.4 | 2.1 | 2.2 | 2.3 | 2.5 | 2.6 | 2.6 | 3.1 | 3.7 | 4.7 | 5.9 | 7.3 | 8.3 | 8.7 | 8.9 | 9.2 |
| Ta.IV.ii.a.1.5 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.1.12 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.3.1 | 1.1 | 1.3 | 1.4 | 2.1 | 2.2 | 2.4 | 2.5 | 2.6 | 2.6 | 3.1 | 3.7 | 4.5 | 5.9 | 7.3 | 8.3 | 8.7 | 8.9 | 9.2 |
| Ta.IV.ii.a.3.2 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.3.6 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.3.7 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.3.10 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.3.11 | 1.1 | 1.3 | 1.4 | 2.2 | 2.3 | 2.4 | 2.5 | 2.6 | 2.6 | 3.2 | 3.7 | 4.5 | 5.5 | 5.9 | 7.3 | 7.7 | 8.5 | 8.9 |
| Ta.IV.ii.a.3.13 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.3.14 | 1.1 | 1.3 | 1.4 | 2.2 | 2.3 | 2.4 | 2.5 | 2.6 | 2.6 | 3.2 | 3.7 | 5.5 | 5.9 | 7.3 | 8.5 | 8.7 | 8.9 | 9.2 |
| Ta.IV.ii.a.3.17 | 1.1 | 1.3 | 1.4 | 2.2 | 2.3 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.3.18 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.4.1 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.4.2 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.4.3 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.4.5 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.4.7 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.4.14 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.4.15 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.4.16 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.4.17 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.4.18 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.6.1 | 1.1 | 1.3 | 1.4 | 2.1 | 2.3 | 2.4 | 2.5 | 2.6 | 2.6 | 3.2 | 3.7 | 5.5 | 5.9 | 7.3 | 8.5 | 8.7 | 8.9 | 9.2 |
| Ta.IV.ii.a.6.2 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.6.3 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.6.4 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.6.5 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.6.6 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.6.7 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.6.8 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.6.9 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.6.11 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.6.12 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.6.13 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.6.14 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.6.15 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.6.17 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.11.2 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.11.3 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.11.5 | 1.1 | 1.3 | 1.4 | 2.2 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.11.6 | 1.1 | 1.3 | 1.4 | 2.2 | 2.2 | 2.4 | 2.5 | 2.6 | 2.6 | 3.1 | 3.7 | 4.3 | 5.9 | 7.3 | 8.3 | 8.5 | 8.7 | 8.9 |
| Ta.IV.ii.a.11.7 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.11.8 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.11.9 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.11.10 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.11.13 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.11.15 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.11.17 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.12.1 | 1.1 | 1.3 | 1.4 | 2.1 | 2.2 | 2.4 | 2.5 | 2.6 | 2.6 | 3.2 | 3.7 | 5.5 | 5.9 | 7.3 | 8.5 | 8.7 | 8.9 | 9.2 |
| Ta.IV.ii.a.12.3 | 1.1 | 1.3 | 1.4 | 2.1 | 2.2 | 2.4 | 2.5 | 2.6 | 2.6 | 3.2 | 3.7 | 5.5 | 5.9 | 7.3 | 8.5 | 8.7 | 8.9 | 9.2 |
| Ta.IV.ii.a.12.4 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.12.5 | 1.1 | 1.3 | 1.4 | 2.1 | 2.2 | 2.4 | 2.5 | 2.6 | 2.6 | 3.2 | 3.7 | 5.5 | 5.9 | 7.3 | 8.5 | 8.7 | 8.9 | 9.2 |
| Ta.IV.ii.a.12.6 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.12.7 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.12.8 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.12.9 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.12.11 | 1.1 | 1.3 | 1.4 | 2.1 | 2.2 | 2.4 | 2.5 | 2.6 | 2.6 | 3.2 | 3.7 | 5.5 | 5.9 | 7.3 | 8.5 | 8.7 | 8.9 | 9.2 |
| Ta.IV.ii.a.12.13 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |
| Ta.IV.ii.a.12.14 | 1.1 | 1.3 | 1.4 | 2.1 | 2.2 | 2.4 | 2.5 | 2.6 | 2.6 | 3.1 | 3.7 | 5.5 | 5.9 | 7.3 | 8.5 | 8.7 | 8.9 | 9.2 |

During the vegetative stage, plants were sampled for PCR analysis to identify homozygous lines. With three selected homozygous lines, 6 field trials were conducted during 2009 and 2011 at Monte Buey (Córdoba, 2009), Gutenberg (Córdoba, 2010), Camet (Buenos Aires, 2011), Corral de Bustos (Córdoba, 2011), Daireaux (Buenos Aires, 2011), and Villa Saboya (Buenos Aires, 2011).

Trials included three homozygous lines (events) and the wild type (parental control). Yield data were analyzed using ANOVA with a completely randomized block design at a 95% confidence level using Infostat (Di Rienzo et al., 2010). In this analysis the data were pooled among sites, and sources of variation included genotypes, environments and the genotype by environment interaction. Means were compared a posteriori with a Fisher lest significant test (α=0.05).

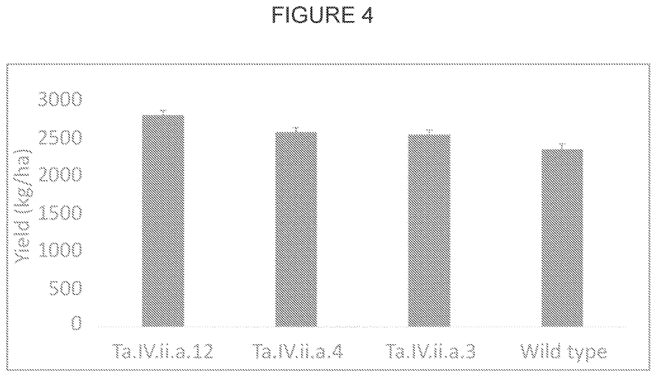

Analysis of variance show statistically significant differences for the effect of genotype (p=0.0003, FIG. 4). In addition, a statistically significant genotype by environment interaction was detected (p<0.0001). This interaction is explained by the crossover of line Ta.IV.ii.a.4 and the control yields at one site (FIG. 4). In the combined analysis all transgenic lines presented higher yield than the parental control. Among lines, Ta.IV.ii.a.12 presented the highest mean yield at all sites. This transgenic line was selected for further characterization and was renamed with the code: IND-ØØ412-7.

### Example 3: Characterization of DNA Sequences in Wheat Event IND-ØØ 412-7

Due to the complex nature of the wheat genome, a strategy to reduce the background genomic information in order to elucidate the insertion structure, sequence and its flanking sequences in the wheat genome was designed. The carrier chromosome was identified using DAT technology platform (Diversity Arrays Technology, Jaccoudy et al., 2001). Once the HaHB4 carrier chromosome was identified, it was isolated applying a chromosome sorting technique (Doležel et al., 1989; Jan Vra'na et al., 2000; Suchánková et al., 2006; Šimková et al., 2008). The sequencing strategy to identify the insertion region of IND-ØØ412-7 in the isolated chromosome consisted of a combined NGS approach using two complementary sequencing technologies: Illumina and Pacific Biosciencies. Illumina produced high-throughput short reads of 100 bp from both sides of a 500 bp-fragment sequencing library. On the other hand, PacBio produced low-throughput single molecule reads from a 15-20 kb-fragment library.

As the size of the insert increases, the likelihood of the polymerase passing over the sequence more than once decreases. For this reason, larger sequences possess a higher error rate than smaller ones. To solve this problem, special algorithms that correct these sequences using smaller sequences obtained by Illumina have been developed. Using this algorithm, PacBio RSII sequences are corrected and a precise and reliable assembly of the sample is obtained (Koren et al., 2012).

### a) Southern Blot Analysis.

The copy number of genes of interest was determined in homozygous IND-ØØ412-7 plants by Southern blot analysis.

Leaf tissue from greenhouse- or field-grown homozygous IND-ØØ412-7 plants or Cadenza plants were used for DNA isolation. One gram of leaf tissue from either IND-ØØ412-7 or Cadenza was flash frozen using liquid nitrogen and ground into fine powder using a pre-chilled pestle and mortar. DNA was extracted with Qiagen DNeasy Maxi Prep Kit following manufacturer's protocol. After elution, the DNA was precipitated by adding 1/10 volume of 3M sodium acetate and 2-3 volumes of 100% ethanol. The pellet was washed with 70% ethanol and suspended in 80 μL of 1×TE buffer. The DNA was quantified using a QuBit fluorometer. The concentration of DNA for IND-ØØ412-7 was 1100 ng/μL and that of Cadenza was 900 ng/μL.

Genomic DNA from event IND-ØØ412-7 was digested with three enzymes: HindIII, BamH1 and AseI. For each 50 μL digestion reaction, 5 μg genomic DNA was mixed with either HindIII, BamHI or AseI enzymes at concentrations of 10 U/μg of DNA. The samples were digested overnight (~16 hours) at 37° C. For digests of the control plasmid, 100-200 picograms of plasmid DNA were used.

The restriction digests of IND-ØØ412-7 and Cadenza genomic DNA were loaded into a 0.7% agarose gel along with DIG labeled Molecular Weight Marker VII (Roche Cat No. 1669940910). The samples were run at 50 V overnight. The gel was incubated in denaturing buffer twice for 30 minutes each time. The denatured gel was washed in transfer buffer for 15 minutes prior to alkaline transfer (outlined below).

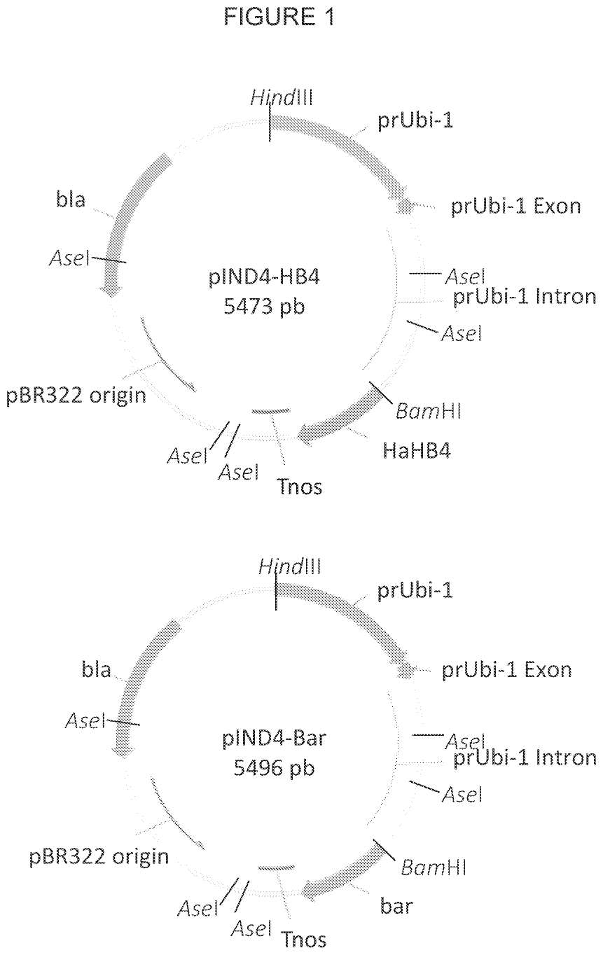

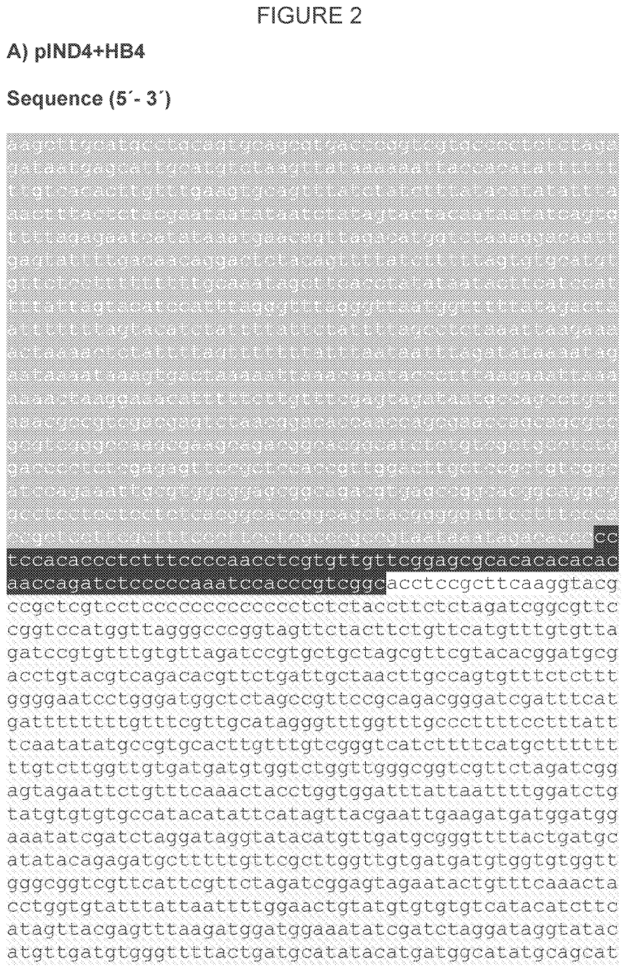

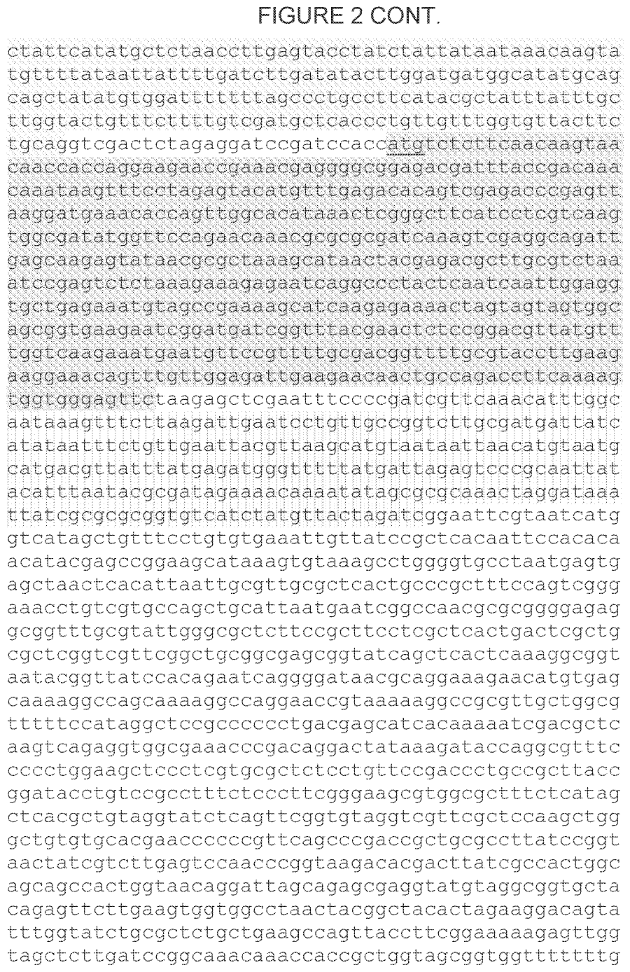

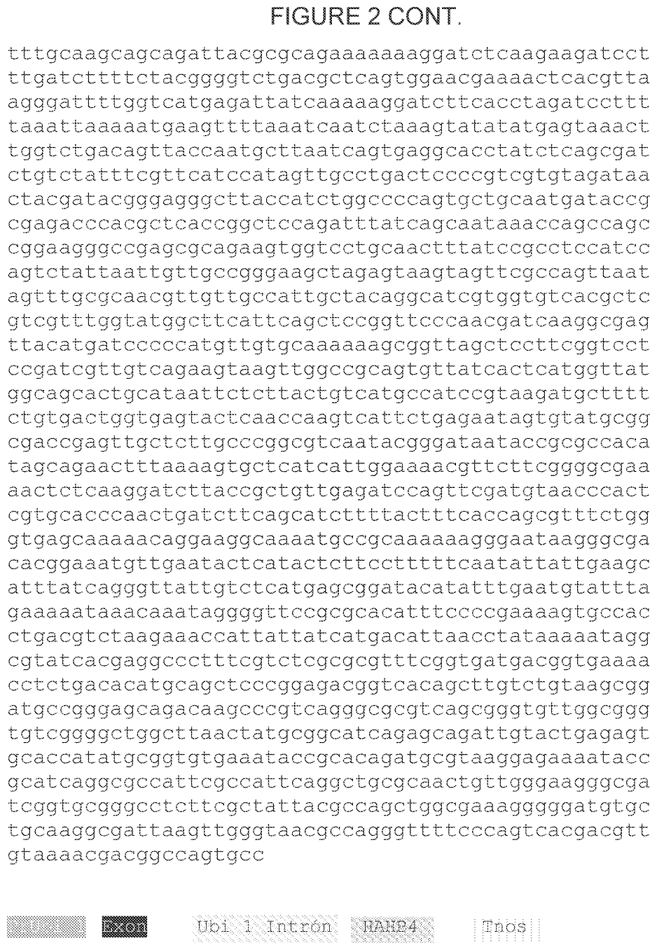

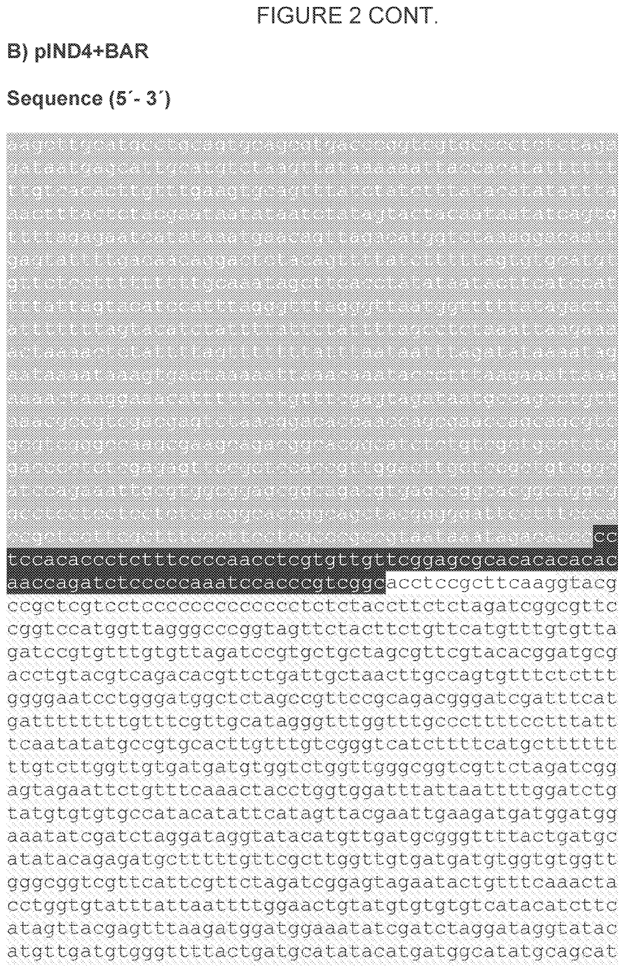

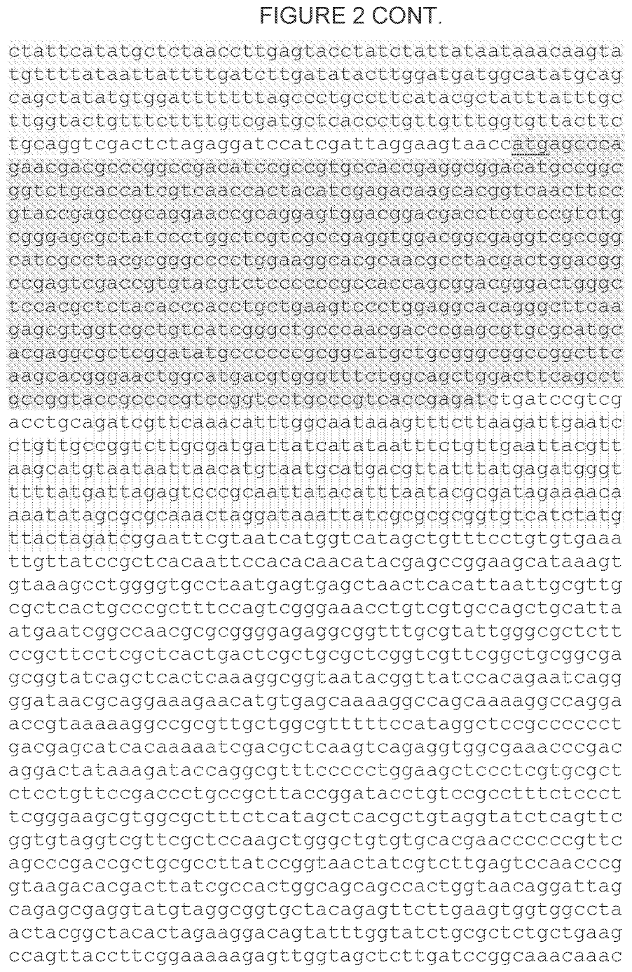

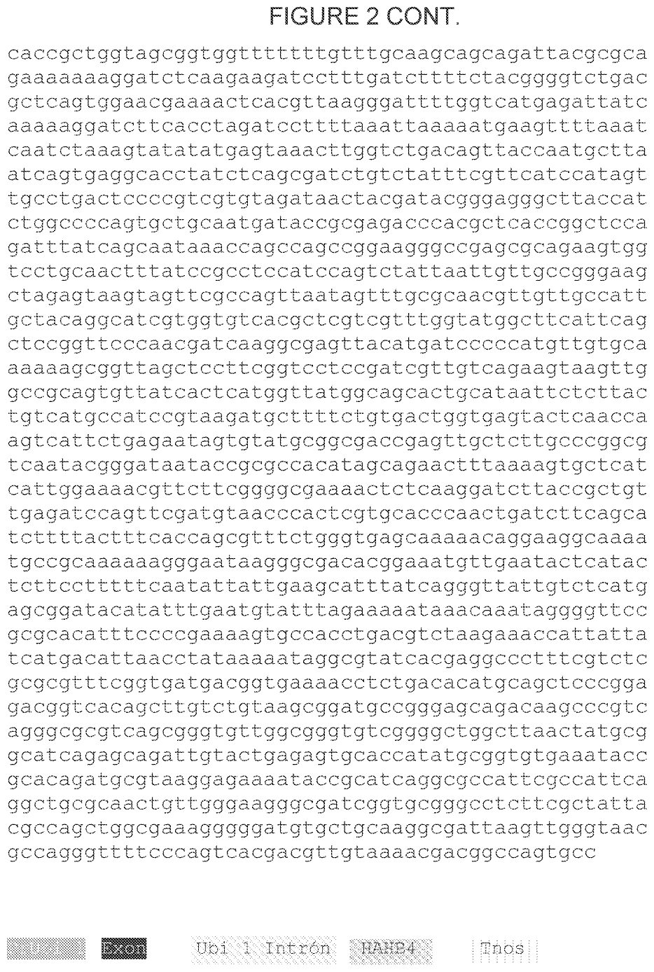

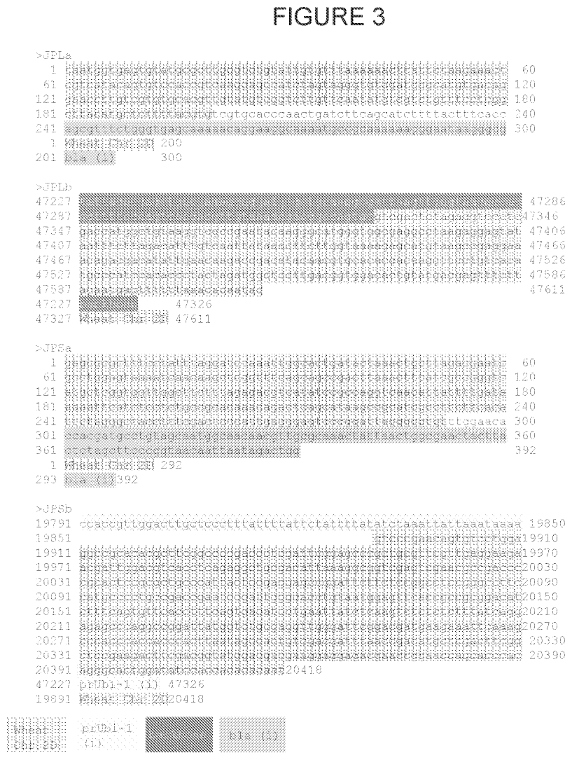

There is one HindIII site in the pIND-4 plasmid, located very close to 5′ prUBI-1 extreme. No other HindIII site was present in the plasmids (FIG. 1). Assuming the occurrence of a single, intact inserts containing the sequence of interest in the genome of IND-ØØ412-7, the minimum fragment size of hybridization of the HaHB4 probe would be 2.842 kbp and that of the bar probe would be 2.860 kbp.

Additionally, there is one BamHI site in the pIND-4 plasmid, located very close to initiation codon of HaHB4 and bar CDSs. No other BamHI sites are present in the plasmids (FIG. 1). Again, assuming the occurrence of a single, intact inserts containing the sequence of interest in the genome of IND-ØØ412-7, the minimum fragment size of hybridization of the HaHB4 probe would be 825 bp and that of the bar probe would be 843 bp.

Finally, there are five AseI restriction sites in the plasmid (FIG. 1), two within of the sequence of interest, in the UBI-1 intron, and three in the vector, two close to the end of Tnos and the other inside of bla gene. Complete AseI digestion in the insert should release a DNA segment of 1.313 kbp long that contains the binding target for the HaHB4 probe. The bar probe will detect a DNA fragment of 1.331 kbp. This consideration is assuming a single intact insertion.

Molecular probes for HaHB4 and bar CDSs were synthesized following the procedure outlined in the Roche PCR DIG Probe Synthesis Kit (Cat. No. 11636090910) and using oligonucleotides mentioned in Table 3.

TABLE 3
List of primers used for the preparation of the
probes used in Southern blot analyses.

| | | Hybridi- | | |
|---|---|---|---|---|
| | Size | zation | Primer | |
| Probe | (bp) | (° C.) | Type | Sequence |
| HB4 | 200 | 45 | Forward | GGGCTTCATCCTGGTCAAGTGGC |
| | | | Reverse | TCTTGATGCTTTTCTGCTACAT |
| | | | | TTCTCAGC |
| Bar | 282 | 55 | Forward | CACGCAACGCCTACGACTGGAC |
| | | | | GGCC |
| | | | Reverse | GTACCGGCAGGCTGAAGTCCAG |
| | | | | CT |

Alkaline transfer of DNA from the agarose gel was performed using the Turboblotter-Rapid downward transfer system (Whatman). The DNA was transferred to a 12×21 cm Nylon membrane (Nytran™ Super Charge, Sigma-Aldrich Co., St. Louis, MO) for 4 hours. The membrane was washed in neutralizing buffer (0.2M sodium phosphate, pH 6.8). The DNA was permanently cross-linked to the membrane by Ultraviolet Cross linker (CL-1000) with 2 exposures of 1500 mJ.

The membrane was incubated in 50 mL of Roche DIG EasyHyb hybridization buffer (Cat. no. 11 603 558 001) at pre-calculated hybridization temperatures (45° C., 55° C. for bar gene and HaHB4 gene probes respectively) on an orbital shaker.

Aliquots of 35 μL and 45 μL bar and HaHB4 probes, were diluted by adding 65 and 55 μL, respectively, of 1×TE buffer. The probe solutions were incubated at 95° C. for 10 min and cooled to 4° C. for 2 min. They were added to 8.75 mL of DIG hybridization buffer and poured to the bottom of the hybridization bottle. The membranes were incubated at the described hybridization temperatures for 16 hours in a hybridization oven (VWR scientific products) with an orbital shaker.

After hybridization, the membranes were washed with washing buffer according to instructions provided with the Roche, DIG Luminescent Detection Kit. After 1 hour of blocking with 1× blocking reagent, the membranes were incubated for 30 min in a solution containing 50 mL of 1× blocking reagent and 5 μL of anti-digoxigenin-AP. The membrane was washed twice with washing buffer for 30 minutes each time and finally treated with detection buffer for 5 minutes. At this point the membrane was placed in a KPL Hybridization Bag (KPL Cat. No. 60-00-51). 5 mL of CSPD solution from the DIG Luminescent Detection Kit was applied evenly across the membrane. The membrane was incubated with CSPD solution for 5 minutes at room temperature. The hybridization bag with the membrane was heat sealed and incubated at 37° C. for 15 minutes. The hybridization bag was placed in a cassette with Kodak Biomax Light Film (Cat. No. 178 8207) in dark room and exposed for 20 minutes. The films were developed at dark using a Konika QX-60A X-ray film processor. Subsequent exposures were made at 1 hour or 2 hours as required.

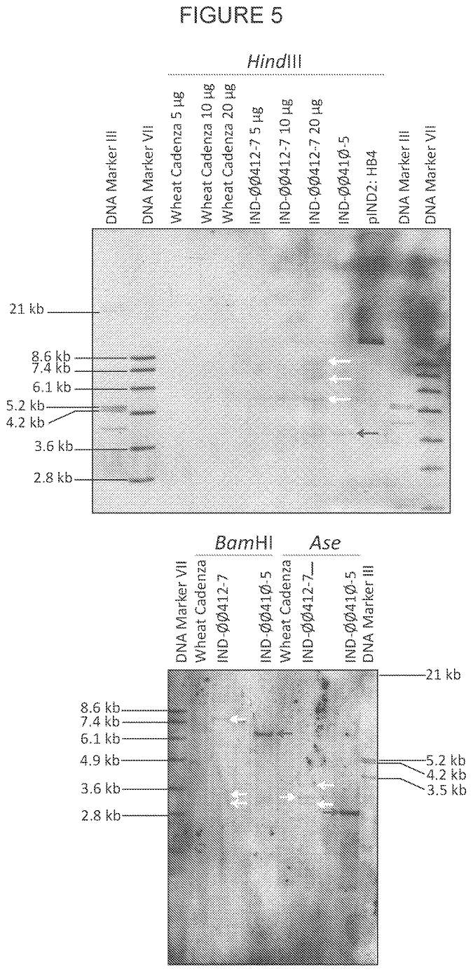

The analysis of hybridization bands obtained with HaHB4 probe allowed to assume the presence of three copies of this CDS (FIG. 5).

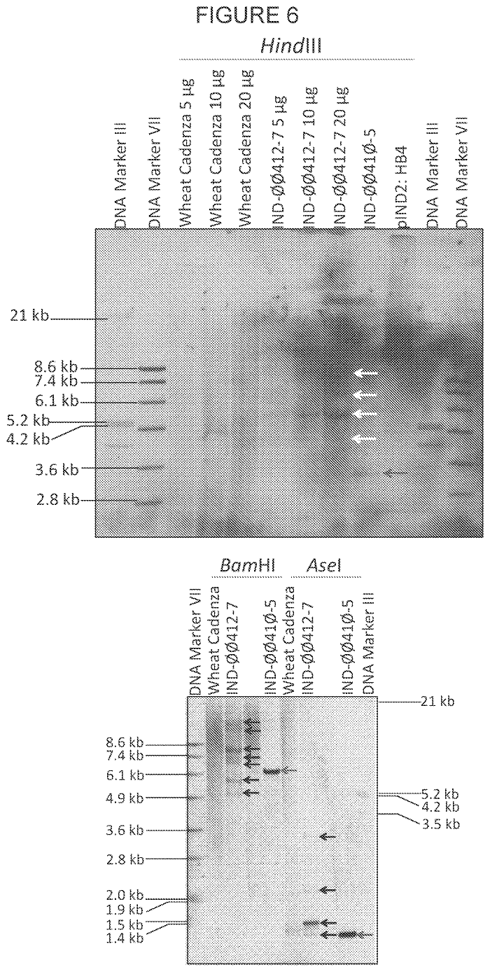

On the other hand, the analysis of hybridization bands obtained with bar probe allowed us to assume the presence of seven copies of this CDS with BamHI (FIG. 6). However, when considering HindIII and AseI the number is lower, indicating possible internal DNA rearrangements (FIG. 6).

In brief, southern blots results showed a complex insertion pattern with possible internal DNA rearrangements, suggesting that a genomic sequencing approach should be necessary to resolve the insertion structure in IND-ØØ412-7 event.

### b) Identification and Isolation of Chromosome Containing the Insertion Sequences in IND-ØØ412-7

DAIT was used to identify that HaHB4 gene located in chromosome 2D of wheat genome. Each polymorphic DNA fragment (which corresponds to a point on the plate) is actually a molecular marker that may serve for the genotyping of a segregating population of a biparental cross (the presence or absence of the marker in the segregating material) and the detection of associations of these markers with characteristic of agronomic interest (region of the polymorphism). In the case of IND-ØØ412-7 event, this technology was used to genotype the F2 generation of the cross IND-ØØ412-7×Bag17, and to search for possible linkage of these markers with HaHB4 transgene. The presence or absence of HaHB4 was determined in each F2 individual and, after genotyping, associations were determined between DArT markers and HaHB4. This transgene presented association with one marker located in the 2D chromosome (http://www.cerealab.unimore.it/). The fact that only one marker presented association to HaHB4 may be because there is not saturated marker map available for this particular chromosome (Jia et al., 2013; http://ccg.murdoch.edu.au/cmap/ccg-live/cgi-bin/cmap/viewer).

The HaHB4 CDS was most probable located in the long arm near the centromere associated to the marker Gpw3105.

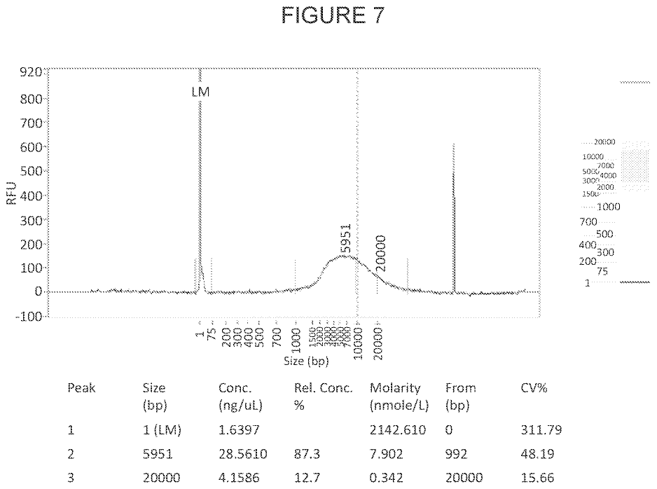

Using this information, 5 μg of chromosome 2D DNA that was suitable for Illumina library preparation were obtained. However, due to the nature of the DNA amplification process post-sorting, the DNA was partially suitable for PacBio libraries (FIG. 7).

Diversity panels were generated using DNA from the IND-ØØ412-7 and wild genotype Cadenza.

Genomic DNA was extracted from seedlings. About 5 ng DNA from each genotype was digested evaluated for 1 hour at 37° C. with 2 U of restriction enzymes (EcoRI, PstI and MspI) in a final volume of 8 μL. After digestion, 2 μL of ligation mixture were added and the sample was incubated for 3 hours at 37° C. The ligation mixture is comprised of 0.2 μL of T4 ligase (New England Biolabs), 0.2 μL of 50 mM ATP, 1.2 μL of MilliQ water (MQ) and 0.1 μL of suitable specific enzyme at a concentration of 50 μmol/μL (MspI) and 5 μmol/μL (EcoRI and PstI). After ligation, the mixture was diluted to a final volume of 500 μL using MQ water, and 2 μL was used as template in a PCR reaction. The reaction was conducted in a final volume of 50 μL, 2 U Red Taq polymerase (Sigma) and primers. After an initial incubation at 95° C. for 3 minutes, the reaction followed the following protocol: 30 cycles of 94° C. for 30 seconds, 60° C. for 45 seconds and 72° C. for 1 minute. A final extension was performed at 72° C. for 8 minutes.

The amplicons thus generated were ligated into the PCR2.1TOPO vector (Invitrogen) and subsequently transformed by heat shock cells of E. coli TOP10F ‘(Invitrogen) following manufacturer's instructions. The transformants were selected on media containing ampicillin and X-gal. Individual white colonies (containing the recombinant plasmid) were transferred to a tube by ringing containing 20 μL of 10% glycerol. From each sample in glycerol, 5 μL aliquot was taken and was transferred to a tube containing 45 μL of PCR master mix RedTaq tube (Sigma) and 15 μmol of each primer M13 forward and M13 reverse. The profile cycler used was 1 cycle at 95° C. for 5 minutes; 35 cycles of 94° C. for 30 seconds, 52° C. for 30 seconds and 72° C. for 1 minute. After amplification, PCR products were precipitated using 1 volume of isopropanol at room temperature and washed with 100 μL of 70% ethanol. The ethanol was removed and the products were dried at room temperature.

The products were re-suspended in MQ water, 3×SSC or 1×SSC+0.01% sarkosyl (Sigma) at a final concentration of ~20 ng/μL. The purified fragments were transferred to a 384-well plate (Genetix) and 6 replicates were set for passage in Polysine™ chips (Menzel Glazer) using the Affymetrix™ 417 System. After 1 day of fixation, the chips were processed according to the http://microarrays.org/protocols.html published procedure (2001) with the slight modification that sodium borohydride was used (Sigma) instead of using succinic anhydride and 1-methyl-2-pyrrolidone as blocking solution, prepared in PBS buffer pH 7.4 (137 mM NaCl, 2.7 mM KCl, 10 mM NaHPO, KHHPO 2 mM). The chips were immersed in boiling water for 30 seconds to denature the DNA and then immediately immersed in iced 100% ethanol for 10 seconds. The chips were dried at room temperature.

After PCR amplification, the products were purified using columns (Qiagen) or precipitated by addition of isopropanol to remove excess dNTPs. The Cy3 and Cy5 (Pharmacia) fluorescent dyes were incorporated using labeling kit Deca-random-prime DNA (Fermentas) according to the manufacturer's instructions.

The samples labeled with Cy3 and Cy5 (5 μL of each) dyes were mixed with 2 μL of a solution of 20 μg/μL DNA from herring sperm (Sigma) dissolved in Express Hybridization solution (Clontech) and denatured at 96° C. for 3 minutes. The denatured probes were mixed with 10 to 15 μL of hybridization solution, transferred directly to the surface of the microarray, and covered with a glass (24×24 mm, Mediglass). The chips were immediately placed in a humidification chamber at 65° C. overnight for hybridization. After hybridization, the chips were washed with 1×SSC with 0.1% SDS for 5 min, 1×SSC for 2 minutes, 0.2×SSC for 2 minutes and 20 seconds 0.02×SSC.

The chips were scanned using the Affymetrix 418™ scanner. The intensity of each signal was analyzed using the Scanalyse v.2.44 (Standford University) and GenePix Pro v.3 (Axon instruments) program.

### c) Junction Sequence Analysis and Structure of the Insertion in IND-ØØ412-7 Using Illumina and PacBio Sequencing.

Results from Southern blot analysis suggested a complex pattern of transgenic insertion. Therefore, isolated chromosome 2D DNA library was sequenced using Illumina Hiseq platform. We produced more than 1.2 billion reads in 2×100 bp paired-end configuration representing more than 124 Gb of sequencing data. Sequencing coverage of the whole chromosome was estimated between 40 and 50× based on not redundant mappable reads on single copy genes located in chromosome 2D such as PpoD1b (He et al, 2009). Sample enrichment in chromosome 2D was verified by mapping reads to known single copy genes located outside the chromosome such LOXs (Feng et al., 2012). In these cases, coverage was estimated between 1 and 5×, thereby confirming a highly enriched DNA in chromosome 2D sequences.

Read mapping on plasmids used for transformation allowed us to verify the Southern blot results. It was concluded that we had obtained 3 complete or nearly complete copies of HaHB4 CDS based on 100× coverage and 8 complete or nearly complete copies of bar CDS based on 400× coverage.

Several other plasmid elements were identified with coverage up to 1200×. Junction Sequence Analysis (JSA) protocol (Kovalic et al., 2012) using Illumina reads identified up to 4 different flanking sequences with wheat DNA and several chimeric junction sequences involving plasmid elements. In brief, Illumina data also showed a complex pattern of the insertion event involving internal plasmid rearrangements in IND-ØØ412-7.

Consequently, PacBio long sequencing reads to assist the scaffolding process of the Illumina reads assembly were generated. PacBio long reads would allow reading through complex internal structures and rearrangements.

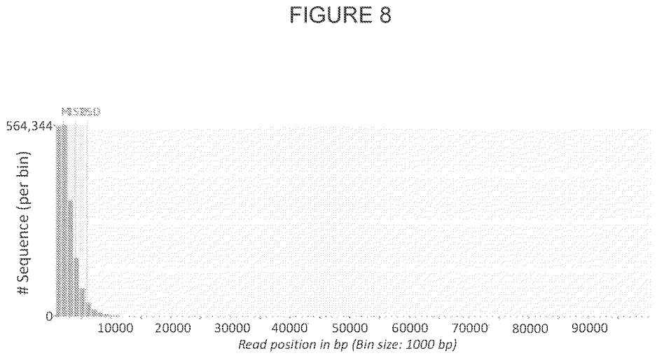

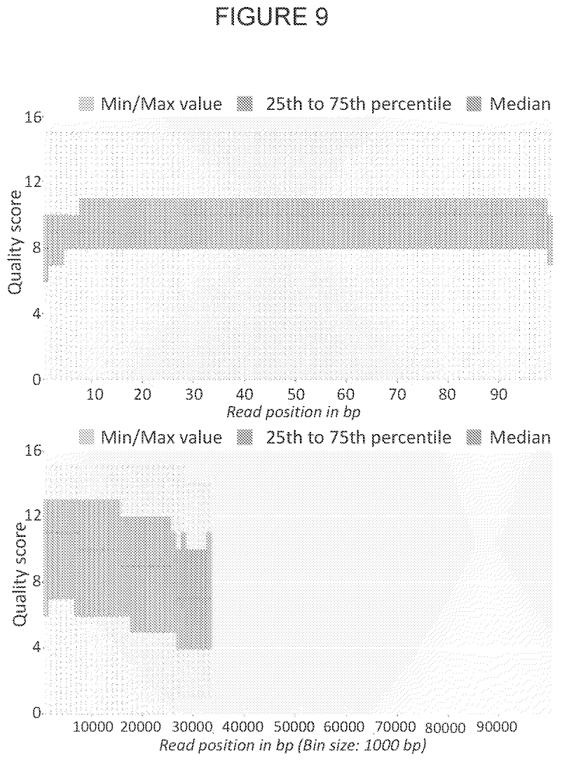

Due to the DNA sample quality obtained after chromosome sorting which allow us to generate a library of around 5 Kb average size, the P4-C2 chemistry of PacBio was used. This chemistry produces an average of 5.5 Kb long reads. 20 SMART cells to generate 1,806,197 reads totaling 3.5 Gb of sequencing data with an average read length of approximately 2 Kb and a maximum of 13 Kb were used. Based on a chromosome 2D size of 800 Mb, a coverage of 4.7× of the whole chromosome was estimated. PacBio statistics are shown in Table 4 and read length distribution and Q scores shown in FIG. 8 and FIG. 9.

TABLE 4
PacBio Statistics.

| Library average size | 5500 bp |
|---|---|
| # smart cells: | 20 |
| # Sequences | 1,806,197 |
| Total bases | 3.513.329.966 bp. (3, 5 Gb.) |
| Mean sequence length | 1.943,32 ± 1.605,46 |
| Minimum length | 50 bp |
| Maximum length | 32,913 |

Based on a combined analysis of sequencing data from Illumina and PacBio the flanking sequences of the insertion were firstly defined.

A set of four parameters as a cut-off to determine the true identity of a flanking sequence was established:
1. Supported by Illumina data,
2. Supported by PacBio data,
3. Supported by Illumina coverage as a single copy, and
4. Supported by PCR amplification from IND-ØØ412-7 DNA.

Four different flanking sequences passed all filters to meet the selected criteria named JPLa, JPLb, JPSa and JPSb.

A similar approach was used to determine all of the internal chimeric plasmid-plasmid junction sequences but considering the Illumina coverage for the individual plasmid elements involved. Internal junction sequences that passed all filters were extended using Illumina data and mapped on the long PacBio reads to produce a final assembly. The quality of the final assembly was checked back to the initial southern blot data and the results were confirmed.

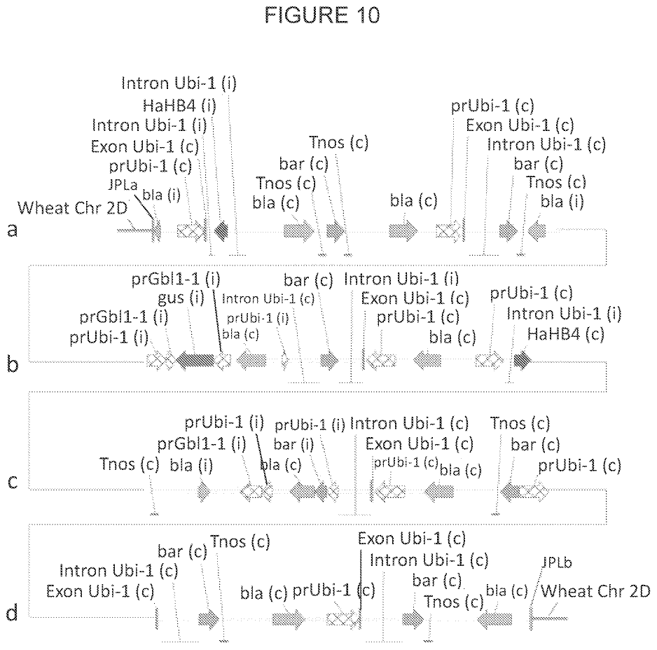

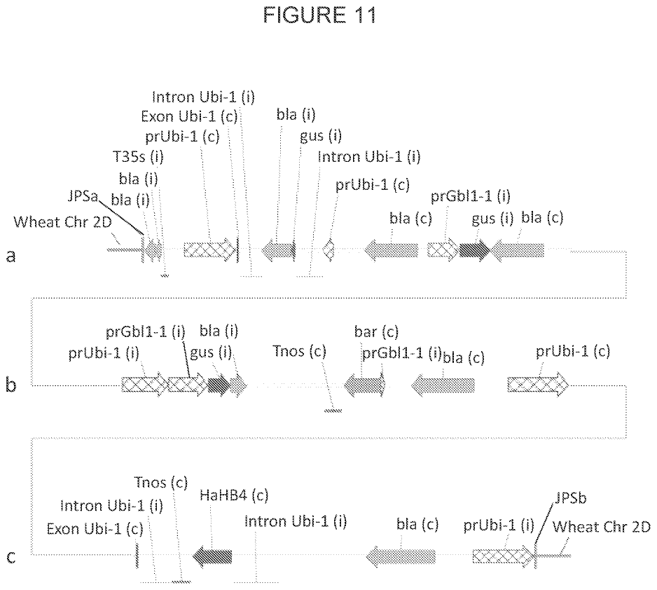

In brief, two different insertions of the plasmids in highly repetitive regions of chromosome 2D in the wheat genome were described. One of the insertions is 47,611 bp long and the other is 20,418 bp long totaling 68,029 bp of insertions in IND-ØØ412-7 event (FIGS. 10 and 11). The insertions accounted for three copies of HaHb4 CDS, seven copies of bar CDS, 16 copies of bla gene and four copies of gus CDS. In the longer insertion, FIG. 10, it is possible to identify a complete HaHB4 coding region (red) downstream of probable functional regulatory elements (panel b). On the other hand, complete bar coding region (light blue) are observed in panels a, b, c and d. In panels (a) and (d) it is possible to identify that the regulatory elements of bar are complete and the right position. The complete copies of Bar CDSs potentially encode functional proteins. As a result, only one HaHB4 copy and one bar copy without complete regulatory elements were identified.

### d) Detection, Structure and Copy Number of Vector Sequences not Related to the Genes of Interest.

From the analysis of the insertion events it was evident the presence of plasmid vector sequences and other coding regions unrelated to the genes and regulatory regions of interest introduced during the wheat transformation process.

Elements of plasmid backbone detected in the insertion of the IND-ØØ412-7 are bla gene conferring ampicillin resistance in bacteria, and bacterial origin of replication of pBR322 plasmid (FIGS. 10 and 11). Regarding bla, it is important to mention that it has no regulatory elements for expression in the wheat as well as none of the described bla copies have been positioned near eukaryotic regulatory elements.

All of the plasmid backbone sequences in IND-ØØ412-7 insertion have a confirmed origin in the vectors pIND4-HB4 and pIND4-Bar used for transformation as demonstrated by the identity of the nucleotide sequences using blast.

However, sequences corresponding to the gus gene (Jefferson et al., 1987) and the promoter of the wheat gbl1 gene (Jones, 2005) leading its expression were detected. These sequences originated in a third plasmid used to monitor the efficiency of transformation; nevertheless, these elements have been incorporated during the integration process. The gus CDS was found in four copies while the gbl1 promoter was found in five copies (FIGS. 10 and 11). This third plasmid would be also a derivative of the series used to incorporated HaHB4 and bar to wheat.

### e) Localization of IND-ØØ412-7 Insertion in the Wheat Genome.

The insertions were located in the wheat chromosome 2D. These insertions were detected in no annotated sequences and it is highly unlikely that they were interrupting coding sequences for the reasons explained below.

All of the flanking wheat sequences corresponded to DNA segments with high similarity to retrotransposons indicating insertion events in highly repetitive DNA regions (Mayer et al, 2014).

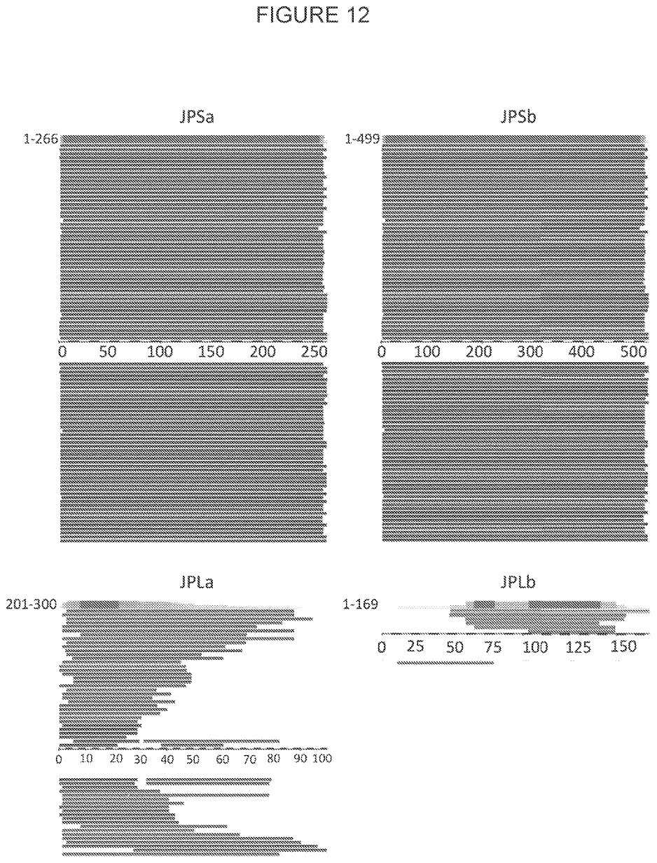

To confirm this, JPs (Junction points) were blasted against IWGSC project database through EnsemblPlants website (http://plants.ensembl.org/Triticum_aestivum/Info/Index). Multiple hits against multiple T. aestivum chromosome sites were observed in each case. Although uneven in some cases, full sequence coverage was observed for each JP (FIG. 12). Because this IWGSC project is still in development, another blast was performed against all remaining sequenced cereals genomes present at EnsemblPlants database (Triticum urartu, Aegilops tauschii y Hordeum vulgare), resulting in the same multiple hit pattern against multiple chromosome sites. Sequences that are present in multiple chromosome sites and conserved in different organisms tend to be repetitive sequences.

JP sequences were also blasted against NCBI non-redundant database. Two of these sequences (JP Short, JPSa and JPSb) matched against known retrotransposon elements (Table 5). While the other two JP sequences (JP Long, JPLa and JPLb) had no significant hits against the NCBI database, they are highly conserved and repeated multiple times across multiple chromosomes in the wheat database, strongly suggesting unknown repeated DNA elements.

TABLE 5
Best hit example of JP sequences blast against NCBI non-redundant database

| | Query | | | Subject | | | Stats | | | |
|---|---|---|---|---|---|---|---|---|---|---|
| Name | Start | End | Name | Start | End | Notes | Score | E-val | % ID | L. |
| JPSa | 1 | 266 | FN564434 | 122244 | 122509 | Retrotransposon | 386 | 1E−92 | 89 | 266 |
| | | | | | | gypsy | | | | |
| JPSb | 1 | 499 | AF326781 | 144816 | 145314 | Transposon | 582 | 7E−146 | 83 | 499 |
| | | | | | | gypsy-like | | | | |
| | | | | | | retrotransposon | | | | |
| | | | | | | Fatima | | | | |
| JPLa | 201 | 300 | IWGSC_CSS_5AS | 364 | 463 | unknown repeat | 500 | 4E−15 | 100 | 100 |
| | | | scaff_1551050 | | | element | | | | |
| JPLb | 1 | 169 | IWGSC_CSS_5DS | 2630 | 2797 | unknown repeat | 507 | 2E−15 | 81 | 174 |
| | | | scaff_2755707 | | | element | | | | |

L.: Length

### Stability and Integrity of the IND-ØØ412-7 Insertions

### Inserts Segregation Over Sexual Transfer.

Homozygous plants for the insertion IND-ØØ412-7 were crossed with a commercial variety, Baguette 17, to obtain the F1 generation. After the selfing of F1 plants, 349 F2 seeds were obtained. Plants grown from these seeds were used for segregation analysis. DNA was extracted from young leaves. Prior to DNA extraction, leaf tissue was frozen in liquid nitrogen and processed to a fine powder in a mortar with a pestle or in tubes in a “96 mill” for minipreps. The following techniques were used to extract plant DNA, depending on the amounts of DNA needed for experimental purposes:

### CTAB Method:

Genomic DNA was extracted following a Hexadecyltrimethylammonium bromide (CTAB)-based method (http://irc.igd.cornell.edu/Protocols/DoyleProtocol.pdf). Briefly, 600 μL of CTAB buffer (2% w/v CTAB, 100 mM TrisHCl, 20 mM EDTA, 1.4 M NaCl, and β-mercaptoethanol) and 5 μg RNase A were added to approximately 100 mg of ground leaf tissue and incubated at 55-60° C. for 15-20 minutes with intermittent mixing. 600 μL of chloroform was added to the samples and mixed by hand 2-3 minutes, then centrifuged at 10,000 rpm for 8 minutes. The upper aqueous phase was put into a clean microtube and the DNA was precipitated with 400 μL of isopropanol. The sample was centrifuged at 12,500 rpm for 10 minutes to pellet the precipitated DNA. The DNA pellets were washed with 300 μL of 70% ethanol by centrifuging the samples at 12,500 rpm for 5 minutes. The DNA pellets were air-dried, then resuspended in 100 μL of TE buffer (10 mM TrisHCl, 1 mM EDTA, pH 8.0). All extracted DNA was stored in a 4° C. refrigerator or in a −20° C. freezer.

To ensure the integrity of the samples, end point PCR of a wheat endogenous control was performed with a specific primer. Then, end point PCR was performed using different combinations of primers to determine the presence of HaHB4 and bar CDS. A Chi-square (χ2) analysis was used to evaluate the expected 3:1 segregation (presence or absence of CDSs of interest).

Table 6 summarizes the results obtained by PCR analysis.

TABLE 6
Results of segregation analysis by means of PCR.

| Total | HaHB4+bar+ | HaHB4+bar− | HaHB4−bar+ | HaHB4−bar− |
|---|---|---|---|---|
| 349 | 259 | 0 | 0 | 90 |

Total: number of individuals tested;
HaHB4+bar+: number of individuals positive to HaHB4 and bar CDSs;
bar−HaHB4+: number of individuals positive to HaHB4 CDS;
HaHB4−bar+: number of individuals positive to bar CDS;
HaHB4−bar−: number of individuals negative to HaHB4 and bar CDSs.

Table 7 shows the statistical analysis:

TABLE 7
Statistical analysis of segregation results.

| Expected results | | Observed results | | | |
|---|---|---|---|---|---|
| (No. of plants) | | (No. of plants) | | | |
| + | − | + | − | X² | p-value |
| 261.75 | 87.25 | 259 | 90 | 0.11 | 0.74 |

The value of χ2 (degrees of freedom=1, α=0.05) showed no statistically significant difference between the expected and observed results indicating a Mendelian segregation 3:1 for each of the CDSs analyzed (HaHB4 and bar).

The results obtained in the F2 generation indicate that both transgenes are stably inherited during sexual transfer. In the segregating population, both genes had a 3:1 segregation according to the Mendelian principles. Finally, even though both CDSs come from a process of co-transformation by bombardment, the two genes are linked, as each individual F2 analyzed presents the same results for both CDSs, bar and HaHB4.

The previous analysis was made with oligonucleotides that detect almost the entire CDSs.

After confirmation of the stability of the complete CDSs of bar and HaHB4, it was decided to proceed with the characterization the other genetic elements, or part of them, and flanking sequences. With the aim of detecting the presence/absence of HaHB4, bar and bla transgenes copies in IND-ØØ412-7, oligonucleotides that hybridize with all copies (complete and incomplete) were designed. Also, the four insert-to-plant junctions were detected to determine their segregation in the F2 generation. These studies were made in 92 individuals of the F2 population. The results are shown in Table 8.

TABLE 8
Statistical analysis of segregation results.

| Expected results | | Observed results | | | |
|---|---|---|---|---|---|
| (No. of plants) | | (No. of plants) | | | |
| + | − | + | − | X² | p-value |
| 69 | 23 | 65 | 27 | 0.9275 | 0.336 |

The χ2 value (d.f.=1, α=0.05) in the F2 generation indicated no statistically significant difference between the observed and the expected 3:1 segregation ratio for the data analyzed.

All transgenic plants analyzed presented consistent results for the three elements of IND-ØØ412-7 detected (HaHB4, bar and bla) and for the four insert-to-plant junctions (JPSa, JPSb, JPLa and JPLb). These results support the conclusion that the IND-ØØ412-7 construct resides at a single locus within the wheat genome and is inherited according to Mendelian inheritance principles.

### Example 4: Yield Increase as an Indicator of Abiotic Stress Tolerance

Fifteen field trials were conducted at multiple test sites and planting dates to evaluate yield and yield components of transgenic event IND-ØØ412-7 and the parental non-transgenic control line (cv Cadenza). Sites were located within the major wheat production areas of Argentina, ranged between latitude 32° S and 38° S. The area covers diverse environmental conditions of Argentinean wheat crop production. Trials included representative locations from pampa's wheat regions described by Kugler and Godoy (1974): IIN (7 trials), IIS (6 trials) and IV (two trials). At each site the planting date was adjusted according to farmers' usual practice, starting in May/June at northern sites and in July at southern sites. Planting and harvest dates are reported in Table 9.

TABLE 9
Selected locations for field trials
and dates of planting and harvest.

| | | | Wheat | Planting | |
|---|---|---|---|---|---|
| Site ID | Site location | Province | region | date | Harvest date |
| A12-1 | Monte Buey | Córdoba | IIN | May 31, 2012 | Dec. 26, 2012 |
| A12-2 | Monte Buey | Córdoba | IIN | Jun. 19, 2012 | Dec. 26, 2012 |
| A13 | Monte Buey | Córdoba | IIN | Jun. 14, 2013 | Dec. 12, 2013 |
| D12 | Corral de | Córdoba | IIN | May 30, 2012 | Dec. 12, 2012 |
| | Bustos | | | | |
| D13 | Corral de | Córdoba | IIN | Jun. 17, 2013 | Dec. 17, 2013 |
| | Bustos | | | | |
| F12 | Villa Saboya | Buenos | IIS | Jun. 7, 2012 | Dec. 22, 2012 |
| | | Aires | | | |
| F13 | Villa Saboya | Buenos | IIS | Jun. 26, 2013 | Dec. 12, 2013 |
| | | Aires | | | |
| G12-1 | Carmen de | Buenos | IIS | Jun. 14, 2012 | Jan. 4, 2013 |
| | Areco | Aires | | | |
| G12-2 | Carmen de | Buenos | IIS | Jul. 6, 2012 | Jan. 4, 2013 |
| | Areco | Aires | | | |
| H12 | Daireaux | Buenos | IIS | Jun. 12, 2012 | Dec. 28, 2012 |
| | | Aires | | | |
| H13 | Daireaux | Buenos | IIS | Jun. 27, 2013 | Dec. 19, 2013 |
| | | Aires | | | |
| I12 | Balcarce | Buenos | IV | Jul. 25, 2012 | Jan. 7, 2013 |
| | | Aires | | | |
| I13 | Balcarce | Buenos | IV | Jul. 26, 2013 | Jan. 13, 2014 |
| | | Aires | | | |
| P12 | Landeta | Santa Fe | IIN | Jun. 3, 2012 | —^{a} |
| P13 | Landeta | Santa Fe | IIN | Jun. 10, 2013 | Dec. 11, 2013 |

^{a}Trial lost by flood at maturity

Weather conditions were registered during the growing season for each site. Annual rainfall registered across sites ranged from 424 mm to 1497 mm for trials H13 and G12 respectively (Table 10), indicating a wide weather variation across sites and years. During 2013 rainfall accumulated during the growing period (June-December) registered values under 500 mm for all sites. Trials planted in 2013 had higher probability of drought stress conditions than the trials planted in 2012. For 2012 rainfall values were above 500 mm for the same period at all sites (Table 10).

TABLE 10
Weather data (rainfall) for each site during 2012 and 2013 for the wheat field sites.

| | Site ID ^{a} | | | | | | | | | | | | |
|---|---|---|---|---|---|---|---|---|---|---|---|---|---|
| Month | A12 | A13 | D12 | D13 | F12 | F13 | G12 | H12 | H13 | I12 | I13 | P12 | P13 |
| January | 47 | 36 | 103 | 25 | 110 | 0 | 75.5 | 112 | 32 | 46 | 69 | 98 | 17 |
| February | 214 | 87 | 282 | 159 | 227 | 5 | 211 | 111 | 10 | 66 | 33 | 164 | 106 |
| March | 212 | 68 | 211 | 42.9 | 104 | 96 | 166 | 184 | 46 | 78 | 98 | 211 | 55 |
| April | 9 | 106 | 19 | 83 | 105 | 30 | 57 | 33 | 70 | 46 | 45 | 83 | 112 |
| May | 48 | 44 | 49 | 33 | 56 | 31 | 120 | 166 | 17.5 | 85 | 10 | 44 | 35 |
| June | 0 | 16 | 0 | 24.5 | 0 | 0 | 7 | 0 | 6 | 0 | 23 | 2 | 17 |
| July | 0 | 12 | 0 | 13.5 | 0 | 8 | 11 | 0 | 45 | 0 | 50 | 0 | 5 |
| August | 68 | 0 | 56 | 0 | 48 | 0 | 203 | 135 | 3 | 295 | 17 | 87 | 0 |
| September | 135 | 2.5 | 132 | 4.5 | 55 | 15 | 72.5 | 70 | 36 | 42 | 141 | 115 | 16 |
| October | 321 | 162 | 291 | 126 | 245 | 58 | 285 | 252 | 37.5 | 27 | 50.5 | 236 | 33 |
| November | 73 | 131 | 155 | 184 | 165 | 150 | 98 | 87 | 111 | 77 | 183 | 64 | 294 |
| December | 128 | 97 | 156 | 24.1 | 41 | 54 | 191 | 179 | 11 | 150 | 10 | 314 | 0 |
| June-December ^{b} | 725 | 421 | 790 | 377 | 554 | 285 | 868 | 723 | 250 | 591 | 452 | 818 | 365 |
| Year | 1255 | 762 | 1453 | 720 | 1156 | 447 | 1497 | 1328 | 425 | 912 | 730 | 1418 | 690 |

^{a} A12 = Monte Buey, two planting dates, year 2012; A13 = Monte Buey, year 2013; D12 = Corral de Bustos, year 2012; D13 = Corral de Bustos, year 2013; F12 = Villa Saboya, year 2012; F13 = Villa Saboya, year 2013; G12 = Carmen de Areco, two planting dates, year 2012; H12 = Daireaux, year 2012; H13 = Daireaux, year 2013; I12 = Balcarce, year 2012; I13 = Balcarce, year 2013; P12 = Landeta, year 2012; P13 = Landeta, year 2013.
^{b} Growing period

Field trials were established at each site following a completely randomized block design. Four replications were evaluated at all locations. Plots consisted of seven rows spaced 0.2 m apart and five meters in length. All field trials were bordered with plots of 7 rows and 5 meters' length. Prior to planting, seed pool of IND-ØØ412-7 and Cadenza were tested by PCR to confirm presence and absence of Hahb4 respectively. All seeds were treated with insecticide (Thiamethoxam, 0.4 mL kg⁻¹), and fungicide (Difenoconazole, 0.2 mL kg⁻¹ and Metalaxyl-M, 0.15 mL kg⁻¹) using the protocol indicated on the corresponding product label.

All chemicals applied to crop were performed according to farmer's usual practice. Herbicides applied during fall prior to planting consisted of Gliphosate (2.0 L ha⁻¹) and Metsulfuron-metil (6.8 g ha⁻¹). Fungicide was applied to control foliar fungal diseases at trials P12, D12, A12-1, A12-2, F12, H12, G12-1, G12-2 and I12. Fungicide was sprayed once at each trial when 50% of the plots reached the growth stage 5.1 (Zadoks, 1974). An insecticide spray to control stink bugs (Nezara sp., Piezodorus sp. and Dichelops sp.) was applied at Zadoks growth stage 3.1 on trials A12-1, A12-2, D12, F12, P12. Conversely, trials P13, D13, A13, F13, H13 and 113 were grown without fungicide or insecticide spray due to low pressure of diseases and pests. Fertilizers and chemicals applied at each trial are described in Table 11.

TABLE 11
Fertilization and chemical inputs for pest control at each trial

| | | Growth | | | |
|---|---|---|---|---|---|
| Site ID | Chemical | Stage^{a} | Rate | Units | Purpose |
| A12-1 | Monoammonium | GS 0 | 160 | kg ha⁻¹ | Fertilizer |
| | phosphate^{b} | | | | |
| | Urea^{c} | GS 2.4 | 150 | kg ha⁻¹ | Fertilizer |
| | Cypermethrin 25 EC | GS 3.1 | 25 | g ha⁻¹ | Insecticide |
| | Azoxystrobin + | GS 5.1 | 0.5 | l ha⁻¹ | Fungicide |
| | Cyproconazole | | | | |
| | (Amistar | | | | |
| | Xtra ® 28 SC) | | | | |
| A12-2 | Monoammonium | GS 0 | 160 | kg ha⁻¹ | Fertilizer |
| | phosphate | | | | |
| | Urea | GS 2.3 | 150 | kg ha⁻¹ | Fertilizer |
| | Cypermethrin 25 EC | GS 3.1 | 25 | g ha⁻¹ | Insecticide |
| | Azoxystrobin + | GS 5.1 | 0.5 | l ha⁻¹ | Fungicide |
| | Cyproconazole | | | | |
| | (Amistar | | | | |
| | Xtra ® 28 SC) | | | | |
| A13 | Monoammonium | GS 0 | 160 | kg ha⁻¹ | Fertilizer |
| | phosphate | | | | |
| | Urea | GS 0 | 150 | kg ha⁻¹ | Fertilizer |
| | Urea | GS 2.6 | 150 | kg ha⁻¹ | Fertilizer |
| D12 | Monoammonium | GS 0 | 160 | kg ha⁻¹ | Fertilizer |
| | phosphate | | | | |
| | Urea | GS 2.3 | 150 | kg ha⁻¹ | Fertilizer |
| | Cypermethrin 25 EC | GS 3.1 | 25 | g ha⁻¹ | Insecticide |
| | Azoxystrobin + | GS 5.1 | 0.5 | l ha⁻¹ | Fungicide |
| | Cyproconazole | | | | |
| | (Amistar | | | | |
| | Xtra ® 28 SC) | | | | |
| D13 | Monoammonium | GS 0 | 160 | kg ha⁻¹ | Fertilizer |
| | phosphate | | | | |
| | Urea | GS 0 | 150 | kg ha⁻¹ | Fertilizer |
| | Urea | GS 2.6 | 150 | kg ha⁻¹ | Fertilizer |
| F12 | Monoammonium | GS 0 | 160 | kg ha⁻¹ | Fertilizer |
| | phosphate | | | | |
| | Urea | GS 2.3 | 150 | kg ha⁻¹ | Fertilizer |
| | Cypermethrin 25 EC | GS 3.1 | 25 | g ha⁻¹ | Insecticide |
| | Azoxystrobin + | GS 5.1 | 0.5 | l ha⁻¹ | Fungicide |
| | Cyproconazole | | | | |
| | (Amistar | | | | |
| | Xtra ® 28 SC) | | | | |
| F13 | Monoammonium | GS 0 | 160 | kg ha⁻¹ | Fertilizer |
| | phosphate | | | | |
| | Urea | GS 0 | 150 | kg ha⁻¹ | Fertilizer |
| | Urea | GS 2.6 | 150 | kg ha⁻¹ | Fertilizer |

^{a}GS: Zadoks Scale (Zadoks, 1974)
^{b}Equivalent Grade 11-52-0
^{b}Equivalent Grade 46-0-0
Note:
Dose in the table corresponds to the commercial product

TABLE 11
Fertilization and chemical inputs for pest control at each trial.
Continued.

| | | Growth | | | |
|---|---|---|---|---|---|
| Site ID | Chemical | Stage^{a} | Rate | Units | Purpose |
| G12-1 | Monoammonium | GS 0 | 160 | kg ha⁻¹ | Fertilizer |
| | phosphate | | | | |
| | Urea | GS 2.3 | 150 | kg ha⁻¹ | Fertilizer |
| | Azoxystrobin + | | | | |
| | Cyproconazole | GS 5.1 | 0.5 | l ha⁻¹ | Fungicide |
| | (Amistar | | | | |
| | Xtra ® 28 SC) | | | | |
| G12-2 | Monoammonium | GS 0 | 160 | kg ha⁻¹ | Fertilizer |
| | phosphate | | | | |
| | Urea | GS 2.3 | 150 | kg ha⁻¹ | Fertilizer |
| | Azoxystrobin + | | | | |
| | Cyproconazole | GS 5.1 | 0.5 | l ha⁻¹ | Fungicide |
| | (Amistar | | | | |
| | Xtra ® 28 SC) | | | | |
| H12 | Monoammonium | GS 0 | 160 | kg ha⁻¹ | Fertilizer |
| | phosphate | | | | |
| | Urea | GS 3.1 | 300 | kg ha⁻¹ | Fertilizer |
| | Azoxystrobin + | | | | |
| | Cyproconazole | GS 5.1 | 0.5 | l ha⁻¹ | Fungicide |
| | (Amistar | | | | |
| | Xtra ® 28 SC) | | | | |
| H13 | Monoammonium | GS 0 | 160 | kg ha⁻¹ | Fertilizer |
| | phosphate | | | | |
| | Urea | GS 0 | 150 | kg ha⁻¹ | Fertilizer |
| | Urea | GS 2.6 | 150 | kg ha⁻¹ | Fertilizer |
| I12 | Monoammonium | GS 0 | 160 | kg ha⁻¹ | Fertilizer |
| | phosphate | | | | |
| | Urea | GS 2.3 | 150 | kg ha⁻¹ | Fertilizer |
| | Azoxystrobin + | GS 5.1 | 0.5 | l ha⁻¹ | Fungicide |
| | Cyproconazole | | | | |
| | (Amistar | | | | |
| | Xtra ® 28 SC) | | | | |
| I13 | Monoammonium | GS 0 | 160 | kg ha⁻¹ | Fertilizer |
| | phosphate | | | | |
| | Urea | GS 0 | 150 | kg ha⁻¹ | Fertilizer |
| | Urea | GS 2.6 | 150 | kg ha⁻¹ | Fertilizer |
| P12 | Monoammonium | GS 0 | 160 | kg ha⁻¹ | Fertilizer |
| | phosphate | | | | |
| | Urea | GS 2.3 | 150 | kg ha⁻¹ | Fertilizer |
| | Cypermethrin 25 EC | GS 3.1 | 25 | g ha⁻¹ | Insecticide |
| | Azoxystrobin + | GS 5.1 | 0.5 | l ha⁻¹ | Fungicide |
| | Cyproconazole | | | | |
| | (Amistar | | | | |
| | Xtra ® 28 SC) | | | | |
| P13 | Monoammonium | GS 0 | 160 | kg ha⁻¹ | Fertilizer |
| | phosphate | | | | |
| | Urea | GS 0 | 150 | kg ha⁻¹ | Fertilizer |

Agronomic data were assessed at each field trial. The agronomic variables consisted of early stand (NPL), number of spikes m⁻² (NE), number of kernels by spike-1 (NGE), thousand kernel weight (PMG), test weight (PH), yield (REND), aerial biomass (BMA) and harvest index (IC).

Early stand was assessed by counting emerged plants in a central plot area of 0.6 m² before tillering. NE, BMA, weight of grains (PG), PMG and IC were assessed at harvest on a central plot sample of 0.6 m². PMG was assessed on a sample of 10 g of the grains harvested at each plot. The NGE was calculated according to:

$$NGE = ( PG / NE ) \times 1000 / PMG \qquad Eq . [ l ]$$

PH was assessed with the Argentinean standard protocol used for wheat commercialization (Shopper's grain scale). Yield was assessed on the five central rows of each plot for trials A12-1, A12-2, D12, F12 and H12. In the rest of the trials, yield was assessed on the whole plot (7 rows). Yield was corrected by grain moisture (13.5%) and expressed as kg ha⁻¹.

Data of the transgenic event and the control Cadenza were analyzed using ANOVA with a completely randomized block design at a 95% confidence level using Infostat (Di Rienzo et al., 2010). Comparisons between the transgenic event and Cadenza were performed in a combined site analysis for REND, PMG, PH, NPL, NE, NGE, BMA and IC. In this analysis the data were pooled among sites, and sources of variation included genotypes (transgenic and control), environments and the genotype by environment interaction.

In the combined site analysis, three statistically significant differences were detected out of 8 comparisons between IND-ØØ412-7 and the control (Table 12). A statistically significant difference was detected between IND-ØØ412-7 and the conventional control for REND (p=0.0103), PMG (p=0.0039) and PH (p=0.0430). In addition, no statistically significant trial by genotype interaction was detected.

The REND of IND-ØØ412-7 was 133 kg ha⁻¹ higher than the control, which represents an average yield increase of 5%. Conversely, PMG and PH showed lower values in IND-ØØ412-7 compared with the control. No consistent differences were observed in cycle or biotic stress response between the event and the parental control. Overall, significant differences in yield between IND-ØØ412-7 and the control suggests that the transgenic event set more seeds per unit land area. Smaller seed size might occur due to lower source-to-sink ratio during seed filling that also leads to lower PH. Differences between IND-ØØ412-7 and the control represented 5% and 2% for PMG and PH respectively.

TABLE 12
Comparison between IND-ØØ412-7 and the parental control Cadenza
in a combined site analysis of yield and yield components.

| | | Mean (MSE) ^{b} | | | | |
|---|---|---|---|---|---|---|
| Variable ^{a} | Unit | IND-ØØ412-7 | | Cadenza | | |
| NPL | Plants m⁻² | 225 | (6.7) | 221 | (6.7) | |
| NE | Spikes m⁻² | 412 | (11.1) | 410 | (9.7) | |
| NGE | Kernels | 21.7 | (0.8) | 21.8 | (0.7) | |
| | spike⁻¹ | | | | | |
| PMG | g | 28.3 | (0.8) | 29.7 | (0.7) | * |
| PH | kg hl⁻¹ | 63.7 | (1.1) | 64.8 | (1.0) | * |
| YIELD | kg ha⁻¹ | 2641 | (163) | 2508 | (156) | * |
| BMA | kg ha⁻¹ | 10583 | (743) | 10081 | (750) | |
| IC | — | 0.31 | (0.01) | 0.31 | (0.01) | |

^{a} NPL = plants m²; NE = spikes m²; NG = kernels spike⁻¹; PH = test weight; PMG = thousand kernel weight; REND = yield; BMA = aerial biomass; IC = harvest index. n = 60 for NPL; n = 56 for PMG, PH and REND; n = 52 for NG and NE; n = 24 for BMA and IC.
^{b} SE: standard error of the mean
* Statistically significant differences (α = 0.05) between control Cadenza and event IND-ØØ412-7

1.-24. (canceled)

25. A method for detecting the presence of DNA belonging to wheat event IND-ØØ412-7 in a sample, the method comprising:
(a) comparing a sample that contains wheat DNA with a set of primers that produces a diagnostic amplicon for wheat event IND-ØØ412-7 when used in an amplification reaction of nucleic acid with genomic DNA of wheat event IND-ØØ412-7; and
(b) performing a nucleic acid amplification reaction, thus producing the diagnostic amplicon; and
(c) detecting the diagnostic amplicon.

26.-29. (canceled)

30. The method of claim 25, wherein the performing step is by quantitative real time polymerase chain reaction (qPCR) or end-point PCR.

31. The method of claim 25, wherein the set of primer comprise event-specific primers.

32. The method of claim 25, wherein the diagnostic amplicon comprises the sequence selected from the group consisting of SEQ ID NO: 5 and SEQ ID NO: 6.

33. The method of claim 25, wherein the presence of the diagnostic amplicon indicates the presence of the DNA derived from IND-ØØ412-7 in the sample.

34. The method of claim 25, wherein the diagnostic amplicon for event IND-00412-7 when tested in a DNA amplification method, comprises this amplicon sequences in SEQ ID NOS: 7, 8, 9 and/or 10.


SEQUENCE LISTING (14 sequences)

SEQ ID NO: 1 - DNA, 47126 bp, synthetic construct
  source 1..47126: mol_type = other DNA
tcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcgtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgtaacgtccggagagttcgtaaaccgatcatccgattcttcaccgctgccactactactagttttctcttgatgcttttcggctacatttctcagcacctccaattgattgagtagggcctgattctctttctttagagactcggatttagacgcaagcgtctcgtagttatgctttagcgcgttatactcttgctcaatctgcctcgactttgatcgcgcgcgtttgttctggaaccatatcgccacttgacgaggatgaagcccgagtttatgtgccaactggtgtttcatccttaactcgggtctcgactgtgtctcaaacatgtactctaggaaacttatttgtttgtcggtaaatcgtctccgcccctcgtttcggttcttcctggtggttgttacttgttgaagagacatggatccgggtgtgatcctctatagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggatccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtttgttgaatagaatacaaaccctagaaaaacaaggcacatctcggcgccgccaatagaaggcagaaaccctagccgtcatttttcttgaagaatatagagcgacgtcgataggaagatacttaaatgttgaatccaagcacctagaataatgagttagattagaagaaaactatcaacttcgaaatcaagaagaaatgttttattctgttgcataaaactttcgcgtggagaagctctgttcagagcccgggtagccactgggcacgtctcctagatactggactcatcggacgctctcctcgagccattaatatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctctcctgttccgaccctgccgcttaccggatacctgtccgcctttctcccttcgggaagcgtggcgctttctcatagctcacgctgtaggtatctcagttcggtgtaggtcgttcgctccaagctcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattgatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggctcgctcagtgtgtgtgactctgctggtgctggggaatggagctcggggcaccgtatttgtagcgcgcgcgccgctgtacgtggcgccctggcgcggctacgtggaggggagctgcgtgccagaggggaggcgggccgccacgtcgcacggccgtgcttatcgggccatcggcgaggtgcgtgccagcttgggcgtcgcgtgtcgtggagcgctcctgctcgcggcggagctggttgagtggattgcgatgcggtgcttggtacagagctcgatggacaactcgaggagagcgtccgatgagtccagtatctaggagacgtgcccagtggctacccgggctctgaacagagcttctccacgcgaaagttttatgcaacagaataaaacatttcttcttgatttcgaagttgatagttttcttctaatctaactcattattctaggtgcttggattcaacatttaagtatcttcctatcgacgtcgctctatattcttcaagaaaaatgacggctagggtttctgccttctattggtcattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaatgtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagtcgactcgtagaggatccatcccgattaggaaagtagagcatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaatttataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtacgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggcatgcaagcttgcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcatttatgcagctggcacgacaggtttcccgactggaaagcgggcagtgagcgcaacgcaatttatgtgagttagctcactcattaggcaccccaggctttacactttatgcttccggctcgtatgttaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacacccgaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccatgtctcttcaacaagtaacaaccaccaggaagaaccgaaacgaggggcggagacgatttaccgacaaacaaataagtttcctagagtacatgtttgagacacagtcgagacccgagttaaggatgaaacaccagttggcacataaactcgggcttcatcctcgtcaagtggcgatatggttccagaacaaacgcgcgcgatcaaagtcgaggcagattgagcaagagtataacgcgctaaagcataactacgagacgcttgcgtctaaatccgagtctctaaagaaagagaatcaggccctactcaatcaattggaggtgctgagaaatgtagccgaaaagcatcaagagaaaactagtagtagtggcagcggtgaagaatcggatgatcggtttacgaactctccggacgttatgtttggtcaagaaatgaatgttccgttttgcgacggttttgcgtaccttgaagaaggaaacagtttgttggagattgaagaacaactgccagaccttcaaaagtggtgggagttcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagatagaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgacgctgggccgcctttttacggttcctggccttttgctggccttttgctcacatgttctttcctgcgttatcccctgattctgtggataaccgtattaccgcctttgagtgagctgatagtgtcacgcagatcctatgaaatttggtcgatgattgtcttttatggatccgttcagaccaagtcttcgtgtgtctatgttatcgtgcctttaaattgaatcctttcgatttatgcttctttttgtcagcggtggttgatcttctgatgtgctggttacaaagggtcttagcataacaattttccaattgtctactacaacaagttttgtctaacttcgatgaagcctaggtgatgacgatttcaacttgcttcggtgcttgtaactgttattagatggtctacggatctaaatgtaacttctattatttgttgtgttttttgtattattatgatggatgatgaatagattaaaagatcccgcaaataaaaccttaaatgataaatgtcacacgtcacgtgcatggcactccggccctaatgccttttgtagctattctagtaacgcaaggatagcaaatttccgtgacacgacgcaagtttttgttaaaaataaattaaagcaagaaaattgtcatacttgctaacgaaaattgccatccttgcctattcatcatccatcataataatacaaaaaacacaacaaataatagaagttacatttagatccgtagaccatctaataacagttacaagcaccgaagcaagttggtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaactcaattgtcctttagaccatgtctaactgttcatttatatgattctctaaggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgaataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggcatgcaagcttctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggcatgcaagcttgcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggatccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatcgattaggaagtaaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgc

SEQ ID NO: 2 - DNA, 19598 bp, synthetic construct
  source 1..19598: mol_type = other DNA
ttggaacaccacgatgcctgtagcaatggcaacaacgttgcgcaaactattaactggcgaactacttactctagcttcccggtaacaattaatagactggatggaggcggataaagttgtaggaccacttctgcgctcggcccttccggctggctggtttattgctgataaatctggagccggactctaggcttcccgggcaacaaattaattagactggatggagggataaagtttgcagaccactttctgcgctcggcccttccgctgggctgttctatgcccgctgaaatcaccagtctctctctacaaatctatctctctctataataatgtgtgagtagttcccagataagggaattagggttcttatagggtttcgctcatgtgttgagcatataagaaacccttagtatgtatttgtatttgggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcgtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactgttattgccgggaaaagtgtacgtatcaccgtttgtgtgaacaacgaactgaactggcagactatcccctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagaagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaaaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaagccaatagaaggcagaaaccctagccgtcatttttcttgaagaatatagagcgacgtcgataggaagatacttaaatgttgaatccaagcacctagaataatgagttagattagaagaaaactatcaacttcgaaatcaagaagaaatgttttattctgttgcataaaactttcgcgtggagaagctctgttcagagcccgggtagccactgggcacgtctcctagatactggactcatcggacgctctcctcgagttgtccatcgagctctgtaccaagcaccgcatcgcaatccactcaaccagctccgccgcgagcaggagcgctccacgacacgcgacgcccaagctggcacgcacctcgccgatggcccgataagcacggccgtgcgacgtggcggcccgcctcccctctggcacgcagctcccctccacgtagccgcgccagggcgccacgtacagcggcgcgcgcgctacaaatacggtgccccgagctccattccccagcaccagcagagtcacacacactgagcgagctaaaccatcaggaagtgatggagcatcagggcggctatacgccatttgaagccgatgtcacgccgtatgttattgccgggaaaagtgtacgtatcaccgtttgtgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtctgaatagaatacaaaccctagaaaaacaaggcacatctcggcgccgccaatagaaggcagaaaccctagccgtcatttttcttgaagaatatagagcgacgtcgataggaagatacttaaatgttgaatccaagcacctagaataatgagttagattagaagaaaactatcaacttcgaaatcaagaagaaatgttttattctgttgcataaaactttcgcgtggagaagctctgttcagagcccgggtagccactgggcacgtctcctagatactggactcatcggacgctctcctcgagttgtccatcgagctctgtaccaagcaccgcatcgcaatccactcaaccagctccgccgcgagcaggagcgctccacgacacgcgacgcccaagctggcacgcacctcgccgatggcccgataagcacggccgtgcgacgtggcggcccgcctcccctctggcacgcagctcccctccacgtagccgcgccagggcgccacgtacagcggcgcgcgcgctacaaatacggtgccccgagctccattccccagcaccagcagagtcacacacactgagcgagctaaaccatggtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacgctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttcactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcaggtcgacggatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggatccagctccgccgcgagcaggagcgctccacgacacgcgacgcccaagctggcacgcacctcgccgatggcccgataaggcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcgtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattccccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcttagaactcccaccacttttggaactcccaccacttttgaaggtctggcagttgttcttcaatctccaacaaactgtttccttcttcaaggtacgcaaaaccgtcgcaaaacggaacattcatttcttgaccaaacataacgtccggagagttcgtaaaccgatcatccgattcttcaccgctgccactactactagttttctcttgatgcttttcggctacatttctcagcacctccaattgattgagtagggcctgattctctttctttagagactcggatttagacgcaagcgtctcgtagttatgctttagcgcgttatactcttgctcaatctgcctcgactttgatcgcgcgcgtttgttctggaaccatatcgccacttgacgaggatgaagcccgagtttatgtgccaactggtgtttcatccttaactcgggtctcgactgtgtctcaaacatgtactctaggaaacttatttgtttgtcggtaaatcgtctccgcccctcgtttcggttcttcctggtggttgttacttgttgaagagacatggatccgagtcgacctgcagaagtaacaccaaacaacagggtgacactcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaacgctgtcttgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaacttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtttgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccctttattttattctattttatatctaaattattaaataaaaaaactaaaatagagttttagttttcttaatttagaggcta

SEQ ID NO: 3 - DNA, 5470 bp, synthetic construct
  source 1..5470: mol_type = other DNA
aagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccgatccaccatgtctcttcaacaagtaacaaccaccaggaagaaccgaaacgaggggcggagacgatttaccgacaaacaaataagtttcctagagtacatgtttgagacacagtcgagacccgagttaaggatgaaacaccagttggcacataaactcgggcttcatcctcgtcaagtggcgatatggttccagaacaaacgcgcgcgatcaaagtcgaggcagattgagcaagagtataacgcgctaaagcataactacgagacgcttgcgtctaaatccgagtctctaaagaaagagaatcaggccctactcaatcaattggaggtgctgagaaatgtagccgaaaagcatcaagagaaaactagtagtagtggcagcggtgaagaatcggatgatcggtttacgaactctccggacgttatgtttggtcaagaaatgaatgttccgttttgcgacggttttgcgtaccttgaagaaggaaacagtttgttggagattgaagaacaactgccagaccttcaaaagtggtgggagttctaagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 4 - DNA, 5496 bp, synthetic construct
  source 1..5496: mol_type = other DNA
aagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatcgattaggaagtaaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 5 - DNA, 47611 bp, synthetic construct
  source 1..47611: mol_type = other DNA
taatggtgagtgtatgcgcttgcgtctgtattgtgtttaaaaaactcattctaagaaactcgtcatacagtgtccaccgtcaaggagccatctagtagggtgtggatgggcatgtgacaggaaccttgtcgtgtgcacgttgtatgtcggtcttgttcaatatgtcgtctgtttcctcggcttacatgctcttttacgtgtcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcgtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgtaacgtccggagagttcgtaaaccgatcatccgattcttcaccgctgccactactactagttttctcttgatgcttttcggctacatttctcagcacctccaattgattgagtagggcctgattctctttctttagagactcggatttagacgcaagcgtctcgtagttatgctttagcgcgttatactcttgctcaatctgcctcgactttgatcgcgcgcgtttgttctggaaccatatcgccacttgacgaggatgaagcccgagtttatgtgccaactggtgtttcatccttaactcgggtctcgactgtgtctcaaacatgtactctaggaaacttatttgtttgtcggtaaatcgtctccgcccctcgtttcggttcttcctggtggttgttacttgttgaagagacatggatccgggtgtgatcctctatagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggatccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtttgttgaatagaatacaaaccctagaaaaacaaggcacatctcggcgccgccaatagaaggcagaaaccctagccgtcatttttcttgaagaatatagagcgacgtcgataggaagatacttaaatgttgaatccaagcacctagaataatgagttagattagaagaaaactatcaacttcgaaatcaagaagaaatgttttattctgttgcataaaactttcgcgtggagaagctctgttcagagcccgggtagccactgggcacgtctcctagatactggactcatcggacgctctcctcgagccattaatatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctctcctgttccgaccctgccgcttaccggatacctgtccgcctttctcccttcgggaagcgtggcgctttctcatagctcacgctgtaggtatctcagttcggtgtaggtcgttcgctccaagctcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattgatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggctcgctcagtgtgtgtgactctgctggtgctggggaatggagctcggggcaccgtatttgtagcgcgcgcgccgctgtacgtggcgccctggcgcggctacgtggaggggagctgcgtgccagaggggaggcgggccgccacgtcgcacggccgtgcttatcgggccatcggcgaggtgcgtgccagcttgggcgtcgcgtgtcgtggagcgctcctgctcgcggcggagctggttgagtggattgcgatgcggtgcttggtacagagctcgatggacaactcgaggagagcgtccgatgagtccagtatctaggagacgtgcccagtggctacccgggctctgaacagagcttctccacgcgaaagttttatgcaacagaataaaacatttcttcttgatttcgaagttgatagttttcttctaatctaactcattattctaggtgcttggattcaacatttaagtatcttcctatcgacgtcgctctatattcttcaagaaaaatgacggctagggtttctgccttctattggtcattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaatgtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagtcgactcgtagaggatccatcccgattaggaaagtagagcatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaatttataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtacgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggcatgcaagcttgcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcatttatgcagctggcacgacaggtttcccgactggaaagcgggcagtgagcgcaacgcaatttatgtgagttagctcactcattaggcaccccaggctttacactttatgcttccggctcgtatgttaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacacccgaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccatgtctcttcaacaagtaacaaccaccaggaagaaccgaaacgaggggcggagacgatttaccgacaaacaaataagtttcctagagtacatgtttgagacacagtcgagacccgagttaaggatgaaacaccagttggcacataaactcgggcttcatcctcgtcaagtggcgatatggttccagaacaaacgcgcgcgatcaaagtcgaggcagattgagcaagagtataacgcgctaaagcataactacgagacgcttgcgtctaaatccgagtctctaaagaaagagaatcaggccctactcaatcaattggaggtgctgagaaatgtagccgaaaagcatcaagagaaaactagtagtagtggcagcggtgaagaatcggatgatcggtttacgaactctccggacgttatgtttggtcaagaaatgaatgttccgttttgcgacggttttgcgtaccttgaagaaggaaacagtttgttggagattgaagaacaactgccagaccttcaaaagtggtgggagttcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagatagaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgacgctgggccgcctttttacggttcctggccttttgctggccttttgctcacatgttctttcctgcgttatcccctgattctgtggataaccgtattaccgcctttgagtgagctgatagtgtcacgcagatcctatgaaatttggtcgatgattgtcttttatggatccgttcagaccaagtcttcgtgtgtctatgttatcgtgcctttaaattgaatcctttcgatttatgcttctttttgtcagcggtggttgatcttctgatgtgctggttacaaagggtcttagcataacaattttccaattgtctactacaacaagttttgtctaacttcgatgaagcctaggtgatgacgatttcaacttgcttcggtgcttgtaactgttattagatggtctacggatctaaatgtaacttctattatttgttgtgttttttgtattattatgatggatgatgaatagattaaaagatcccgcaaataaaaccttaaatgataaatgtcacacgtcacgtgcatggcactccggccctaatgccttttgtagctattctagtaacgcaaggatagcaaatttccgtgacacgacgcaagtttttgttaaaaataaattaaagcaagaaaattgtcatacttgctaacgaaaattgccatccttgcctattcatcatccatcataataatacaaaaaacacaacaaataatagaagttacatttagatccgtagaccatctaataacagttacaagcaccgaagcaagttggtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaactcaattgtcctttagaccatgtctaactgttcatttatatgattctctaaggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgaataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggcatgcaagcttctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggcatgcaagcttgcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggatccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatcgattaggaagtaaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcgtcgactctagaggtccctcgaccatggctgtaaggtcgccgaatacaagggcatgggtggcgaggcctaagaggagtataatttcttagacatttgtcaattataaacttcttggtaaaagagcatgtaagccgaggaaacagacgacatattgaacaagaccgacatacaacgtgcacacgacaaggttcctgtcacatgcccatccacaccctactagatggctccttgacggtggacactgtatgacgagtttcttagaatgagttttttaaacacaatac

SEQ ID NO: 6 - DNA, 20418 bp, synthetic construct
  source 1..20418: mol_type = other DNA
gagcgcgctttcctatttaggatccaaattggcactgatactaaactgcttagacgaatcgcctggagtaaaatcaacaagctcggtttcagcagccgacttaaacttcatcgccgggtcatgctcggtggttggcttctttagagacgtcatatccgccaggtcaacattattttgataaaaattcatctcctctgccgcacaaacagactcagcataagccgcatcgccttcttcacattctagggctacctttcgactcccattgagggagtcccggattagggggtgtttggaacaccacgatgcctgtagcaatggcaacaacgttgcgcaaactattaactggcgaactacttactctagcttcccggtaacaattaatagactggatggaggcggataaagttgtaggaccacttctgcgctcggcccttccggctggctggtttattgctgataaatctggagccggactctaggcttcccgggcaacaaattaattagactggatggagggataaagtttgcagaccactttctgcgctcggcccttccgctgggctgttctatgcccgctgaaatcaccagtctctctctacaaatctatctctctctataataatgtgtgagtagttcccagataagggaattagggttcttatagggtttcgctcatgtgttgagcatataagaaacccttagtatgtatttgtatttgggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcgtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactgttattgccgggaaaagtgtacgtatcaccgtttgtgtgaacaacgaactgaactggcagactatcccctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagaagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaaaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaagccaatagaaggcagaaaccctagccgtcatttttcttgaagaatatagagcgacgtcgataggaagatacttaaatgttgaatccaagcacctagaataatgagttagattagaagaaaactatcaacttcgaaatcaagaagaaatgttttattctgttgcataaaactttcgcgtggagaagctctgttcagagcccgggtagccactgggcacgtctcctagatactggactcatcggacgctctcctcgagttgtccatcgagctctgtaccaagcaccgcatcgcaatccactcaaccagctccgccgcgagcaggagcgctccacgacacgcgacgcccaagctggcacgcacctcgccgatggcccgataagcacggccgtgcgacgtggcggcccgcctcccctctggcacgcagctcccctccacgtagccgcgccagggcgccacgtacagcggcgcgcgcgctacaaatacggtgccccgagctccattccccagcaccagcagagtcacacacactgagcgagctaaaccatcaggaagtgatggagcatcagggcggctatacgccatttgaagccgatgtcacgccgtatgttattgccgggaaaagtgtacgtatcaccgtttgtgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtctgaatagaatacaaaccctagaaaaacaaggcacatctcggcgccgccaatagaaggcagaaaccctagccgtcatttttcttgaagaatatagagcgacgtcgataggaagatacttaaatgttgaatccaagcacctagaataatgagttagattagaagaaaactatcaacttcgaaatcaagaagaaatgttttattctgttgcataaaactttcgcgtggagaagctctgttcagagcccgggtagccactgggcacgtctcctagatactggactcatcggacgctctcctcgagttgtccatcgagctctgtaccaagcaccgcatcgcaatccactcaaccagctccgccgcgagcaggagcgctccacgacacgcgacgcccaagctggcacgcacctcgccgatggcccgataagcacggccgtgcgacgtggcggcccgcctcccctctggcacgcagctcccctccacgtagccgcgccagggcgccacgtacagcggcgcgcgcgctacaaatacggtgccccgagctccattccccagcaccagcagagtcacacacactgagcgagctaaaccatggtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacgctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttcactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcaggtcgacggatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggatccagctccgccgcgagcaggagcgctccacgacacgcgacgcccaagctggcacgcacctcgccgatggcccgataaggcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcgtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattccccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcttagaactcccaccacttttggaactcccaccacttttgaaggtctggcagttgttcttcaatctccaacaaactgtttccttcttcaaggtacgcaaaaccgtcgcaaaacggaacattcatttcttgaccaaacataacgtccggagagttcgtaaaccgatcatccgattcttcaccgctgccactactactagttttctcttgatgcttttcggctacatttctcagcacctccaattgattgagtagggcctgattctctttctttagagactcggatttagacgcaagcgtctcgtagttatgctttagcgcgttatactcttgctcaatctgcctcgactttgatcgcgcgcgtttgttctggaaccatatcgccacttgacgaggatgaagcccgagtttatgtgccaactggtgtttcatccttaactcgggtctcgactgtgtctcaaacatgtactctaggaaacttatttgtttgtcggtaaatcgtctccgcccctcgtttcggttcttcctggtggttgttacttgttgaagagacatggatccgagtcgacctgcagaagtaacaccaaacaacagggtgacactcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaacgctgtcttgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaacttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtttgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccctttattttattctattttatatctaaattattaaataaaaaaactaaaatagagttttagttttcttaatttagaggctagtcccgaacagtgtcctggaggccgcacacgcttcggccccgaccctcgattcggagcccgctgcgttcgttgaggaagaacgattggacgtcacctcagaggctgcgacattaaaggcggtcgagtcgaacgccgaccccgcactccgcgctgcccattactccgaggagccggattcttctccggcctccgggccttccatgcccctgccgaccgaatccgattgggcacctgtaatggagttcaccgccgcggacatctttcagtgttcacctttcagtgacatcctgaattatctcaagtctctctctttatcaggagagcccaggccggattatggtctgcgaggttgggattcggacgatgaagaaattcaaagcccacccaccacccacttaatagccactgtcgacgatttaaccgacatgctcgacttcggctccgaagacttcgacggtatggacgacgaaggaggagacgaaccggaaccagcacccacagggcactggatatccaccacacacaat

SEQ ID NO: 7 - DNA, 300 bp, synthetic construct
  source 1..300: mol_type = other DNA
taatggtgagtgtatgcgcttgcgtctgtattgtgtttaaaaaactcattctaagaaactcgtcatacagtgtccaccgtcaaggagccatctagtagggtgtggatgggcatgtgacaggaaccttgtcgtgtgcacgttgtatgtcggtcttgttcaatatgtcgtctgtttcctcggcttacatgctcttttacgtgtcgtgcacccaactgatcttcagcatcttttactttcaccagcgtttctgggtgagcaaaaacaggaaggcaaaatgccgcaaaaaagggaataagggcg

SEQ ID NO: 8 - DNA, 385 bp, synthetic construct
  source 1..385: mol_type = other DNA
tattacgccagctggcgaaagggggatgtgctgcaaggcgattaagttgggtaacgccagggttttcccagtcacgacgttgtaaaacgacggccagtgcgtcgactctagaggtccctcgaccatggctgtaaggtcgccgaatacaagggcatgggtggcgaggcctaagaggagtataatttcttagacatttgtcaattataaacttcttggtaaaagagcatgtaagccgaggaaacagacgacatattgaacaagaccgacatacaacgtgcacacgacaaggttcctgtcacatgcccatccacaccctactagatggctccttgacggtggacactgtatgacgagtttcttagaatgagttttttaaacacaatac

SEQ ID NO: 9 - DNA, 392 bp, synthetic construct
  source 1..392: mol_type = other DNA
gagcgcgctttcctatttaggatccaaattggcactgatactaaactgcttagacgaatcgcctggagtaaaatcaacaagctcggtttcagcagccgacttaaacttcatcgccgggtcatgctcggtggttggcttctttagagacgtcatatccgccaggtcaacattattttgataaaaattcatctcctctgccgcacaaacagactcagcataagccgcatcgccttcttcacattctagggctacctttcgactcccattgagggagtcccggattagggggtgtttggaacaccacgatgcctgtagcaatggcaacaacgttgcgcaaactattaactggcgaactacttactctagcttcccggtaacaattaatagactgg

SEQ ID NO: 10 - DNA, 628 bp, synthetic construct
  source 1..628: mol_type = other DNA
ccaccgttggacttgctccctttattttattctattttatatctaaattattaaataaaaaaactaaaatagagttttagttttcttaatttagaggctagtcccgaacagtgtcctggaggccgcacacgcttcggccccgaccctcgattcggagcccgctgcgttcgttgaggaagaacgattggacgtcacctcagaggctgcgacattaaaggcggtcgagtcgaacgccgaccccgcactccgcgctgcccattactccgaggagccggattcttctccggcctccgggccttccatgcccctgccgaccgaatccgattgggcacctgtaatggagttcaccgccgcggacatctttcagtgttcacctttcagtgacatcctgaattatctcaagtctctctctttatcaggagagcccaggccggattatggtctgcgaggttgggattcggacgatgaagaaattcaaagcccacccaccacccacttaatagccactgtcgacgatttaaccgacatgctcgacttcggctccgaagacttcgacggtatggacgacgaaggaggagacgaaccggaaccagcacccacagggcactggatatccaccacacacaat

SEQ ID NO: 11 - DNA, 23 bp, synthetic construct
  source 1..23: mol_type = other DNA
gggcttcatcctggtcaagtggc

SEQ ID NO: 12 - DNA, 30 bp, synthetic construct
  source 1..30: mol_type = other DNA
tcttgatgcttttctgctacatttctcagc

SEQ ID NO: 13 - DNA, 26 bp, synthetic construct
  source 1..26: mol_type = other DNA
cacgcaacgcctacgactggacggcc

SEQ ID NO: 14 - DNA, 24 bp, synthetic construct
  source 1..24: mol_type = other DNA
gtaccggcaggctgaagtccagct